(19) United States
(12) Patent Application Publication
(10) Pub. No.: US 2016/0009763 A1
(43) Pub. Date: Jan. 14, 2016

(54) PEPTIDE MOLECULAR MATERIALS

(71) Applicant: National Chiao Tung University, Hsinchu (TW)

(72) Inventors: Hsin-Chieh Lin, Hsinchu (TW); Shu-Min Hsu, Hsinchu (TW)

(73) Assignee: National Chiao Tung University, Hsinchu (TW)

(21) Appl. No.: 14/594,392

(22) Filed: Jan. 12, 2015

(30) Foreign Application Priority Data
Jul. 10, 2014 (TW) 103123757

(51) Int. Cl. C07K 7/06; C07K 5/062; C12N 5/00; A61K 47/18; C07K 5/083; C07K 5/065

(52) U.S. Cl. CPC C07K 7/06; C07K 5/0806; C07K 5/06078; C12N 5/0068; A61K 47/183; C07K 5/06043; C07K 5/06034

(57) ABSTRACT

This invention provides a novel peptide molecular material, wherein the molecular structure of the material is a combination of halogen-substituted or unsubstituted aryl and a peptide molecular. This material can self-assemble to form a nanofiber and form a hydrogel. The hydrogel has various properties, including low cytotoxicity, the promotion of cell growth and migration as well as being stable under a physiological condition and a human body temperature.

### CROSS-REFERENCE TO RELATED APPLICATION

This application claims foreign priority under 35 U.S.C. §119(a) to Patent Application No. 103123757, filed on Jul. 10, 2014, in the Intellectual Property Office of Ministry of Economic Affairs, Republic of China (Taiwan, R.O.C.), the entire content of which patent application is incorporated herein by reference.

### BACKGROUND OF THE INVENTION

1. Field of the Invention

The present invention relates to a peptide molecular material. Particularly, the present invention relates to a peptide molecular material that can self-assemble to form a hydrogel in the water.

2. Description of Related Art

The current small molecular peptide hydrogel technology requires that the molecule thereof is capable of self-assembling to generate a nanostructure for fixing water flow. Therefore, a long peptide is usually used to provide sufficient intermolecular force, such that the cost is increased. For example, U.S. Pat. No. 7,884,185 discloses a hydrogel material formed of 20 amino acids. In another small molecular peptide hydrogel technology, relatively simple aryl in combination with a peptide fragment that can biologically react is used to reduce the cost. Similar technologies are disclosed in the representative patents, such as U.S. Patent Publication Nos. 2007/0224273 and 2007/0243255. Among these technologies, the aryl group, fluorenylmethyloxycarbonyl (Fmoc), is a self-assembled group that is now widely used. The self-assembled material from Fmoc can generate hydrogels under a physiological condition. However, Fmoc has hydrogen atoms that are easily dissociate, so that its long-term stability is poor. In addition to Fmoc that exhibits hydrogels with low cytotoxicity, a hydrogel with low cytotoxicity formed by a nucleobase and a peptide fragment is described in such as WO 2012/166705A2. Such hydrogel has development potential. However, the synthesis of a nucleobase requires many synthesis steps, so that the cost is increased and a higher concentration (2 to 3 wt %) for forming gel is necessary.

Thus, there is a need to develop a novel peptide molecular material, which can be synthesized by simple steps, and exhibits high stability, non-cytotoxicity and the effect of promoting tissue growth.

### SUMMARY OF THE INVENTION

The present invention provides a peptide molecular material having a structure represented by the following formula 1:

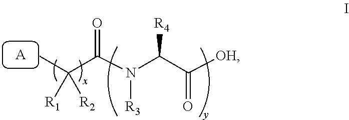

wherein A is an aryl unsubstituted or substituted by one to five halogens, each of the halogens is selected from the group consisting of fluorine, chlorine, bromine and iodine;

R₁ and R₂ are independently selected from the group consisting of a hydrogen atom and substituted or unsubstituted alkyl, and R₁ and R₂ are the same or different; and

R₃ and R₄ are independently selected from the group consisting of a hydrogen atom, C₁-C₁₀ alkyl, C₇-C₁₀ aralkyl, C₂-C₁₀ alkylthioalkyl, C₇-C₁₀ hydroxyaralkyl, C₆-C₁₀ heteroaralkyl, C₂-C₁₀ carboxylalkyl, C₂-C₁₀ guanidinoalkyl and C₁-C₁₀ aminoalkyl, and R₃ and R₄ are the same or different.

In addition, x is an integer of 0 to 10, and when x >1, R₁s or R₂s in formula 1 are the same or different. For example, when x is 2, since there are two repeat units in such position, two R₁ may be the same or different and two R₂ may be the same or different.

Also, y is an integer of 1 to 20, and when y >1, R₃s of R₄s in formula 1 are the same or different. For example, when y is 2, since there are two repeat units in such position, two R₃ may be the same or different and two R₄ may be the same or different.

In one embodiment, A is phenyl substituted by fluorine; R₁ and R₂ are hydrogen atoms; R₃ and R₄ are independently selected from the group consisting of a hydrogen atom and aralkyl; x is 1; and y is 1.

For example, the peptide molecular material of the present invention has a structure represented by the following formula (A):

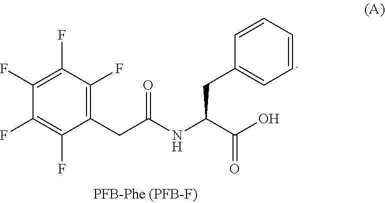 [SMILES: O=C(Cc1c(F)c(F)c(F)c(F)c1F)N[C@@H](Cc1ccccc1)C(=O)O]

In one embodiment, A is phenyl substituted by fluorine; R₁ and R₂ are independently selected from a hydrogen atom; R₃ and R₄ are independently selected from the group consisting of a hydrogen atom, C₁-C₁₀ alkyl, C₇-C₁₀ aralkyl and C₇-C₁₀ hydroxyaralkyl; x is 1; and y is 2.

For example, the peptide molecular material of the present invention has a structure represented by any of the following formulas (B) to (K):

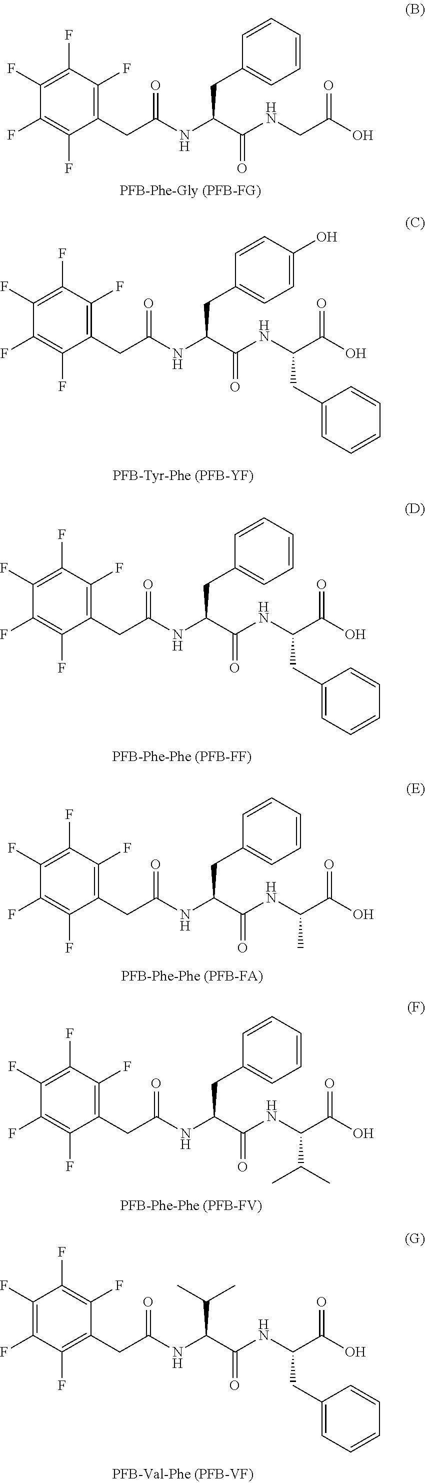 [SMILES: O=C(O)CNC(=O)[C@H](Cc1ccccc1)NC(=O)Cc1c(F)c(F)c(F)c(F)c1F ; O=C(Cc1c(F)c(F)c(F)c(F)c1F)N[C@@H](Cc1ccc(O)cc1)C(=O)N[C@@H](Cc1ccccc1)C(=O)O ; O=C(Cc1c(F)c(F)c(F)c(F)c1F)N[C@@H](Cc1ccccc1)C(=O)N[C@@H](Cc1ccccc1)C(=O)O ; C[C@H](NC(=O)[C@H](Cc1ccccc1)NC(=O)Cc1c(F)c(F)c(F)c(F)c1F)C(=O)O ; CC(C)[C@H](NC(=O)[C@H](Cc1ccccc1)NC(=O)Cc1c(F)c(F)c(F)c(F)c1F)C(=O)O ; CC(C)[C@H](NC(=O)Cc1c(F)c(F)c(F)c(F)c1F)C(=O)N[C@@H](Cc1ccccc1)C(=O)O]

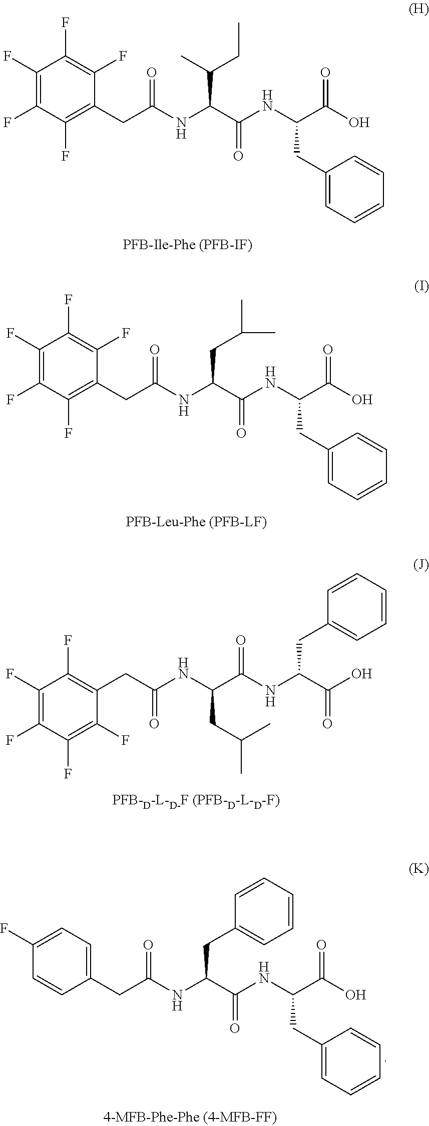 [SMILES: CCC(C)[C@H](NC(=O)Cc1c(F)c(F)c(F)c(F)c1F)C(=O)N[C@@H](Cc1ccccc1)C(=O)O ; CC(C)C[C@H](NC(=O)Cc1c(F)c(F)c(F)c(F)c1F)C(=O)N[C@@H](Cc1ccccc1)C(=O)O ; CC(C)C[C@@H](NC(=O)Cc1c(F)c(F)c(F)c(F)c1F)C(=O)N[C@H](Cc1ccccc1)C(=O)O ; O=C(Cc1ccc(F)cc1)N[C@@H](Cc1ccccc1)C(=O)N[C@@H](Cc1ccccc1)C(=O)O]

In one embodiment, A is phenyl substituted by fluorine; R₁ and R₂ are independently selected from a hydrogen atom; R₃ and R₄ are independently selected from the group consisting of a hydrogen atom, C₁-C₁₀ alkyl, C₇-C₁₀ aralkyl and C₇-C₁₀ hydroxyaralkyl; x is 1; and y is 3.

For example, the peptide molecular material of the present invention has a structure represented by the following formula (L):

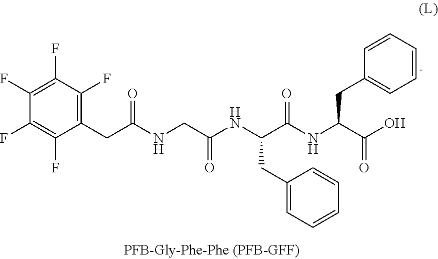 [SMILES: O=C(Cc1c(F)c(F)c(F)c(F)c1F)NCC(=O)N[C@@H](Cc1ccccc1)C(=O)N[C@@H](Cc1ccccc1)C(=O)O]

In one embodiment, A is phenyl substituted by fluorine; R₁ and R₂ are hydrogen atoms; R₃ and R₄ are independently selected from the group consisting of a hydrogen atom, C1-C10 alkyl and C1-C10 aminoalkyl; x is 1; and y is 5.

For example, the peptide molecular material of the present invention has a structure represented by the following formula (M) or (N):

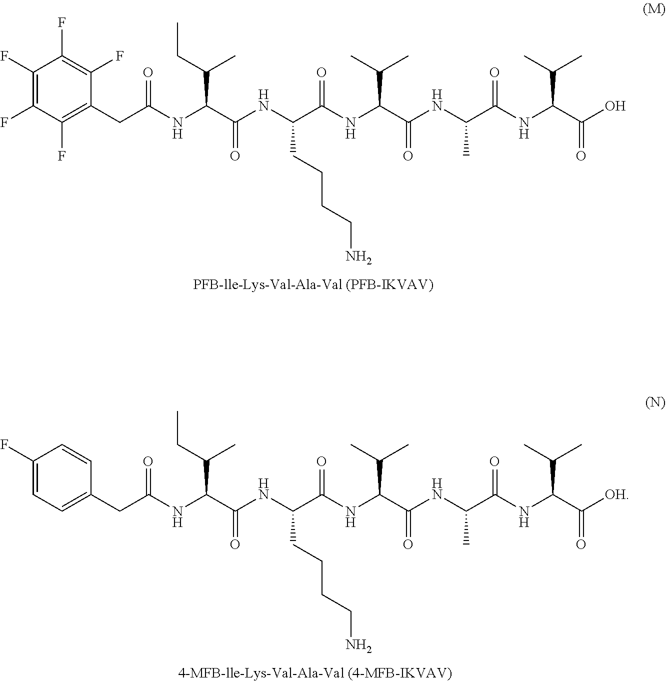 [SMILES: CCC(C)[C@H](NC(=O)Cc1c(F)c(F)c(F)c(F)c1F)C(=O)N[C@@H](CCCCN)C(=O)N[C@H](C(=O)N[C@@H](C)C(=O)N[C@H](C(=O)O)C(C)C)C(C)C ; CCC(C)[C@H](NC(=O)Cc1ccc(F)cc1)C(=O)N[C@@H](CCCCN)C(=O)N[C@H](C(=O)N[C@@H](C)C(=O)N[C@H](C(=O)O)C(C)C)C(C)C]

In one embodiment, A is phenyl; R₁ and R₂ are hydrogen atoms; R₃ and R₄ are independently selected from the group consisting of a hydrogen atom, C1-C10 alkyl and C1-C10 aminoalkyl; x is 0 to 2; and y is 5.

For example, the peptide molecular material of the present invention has a structure represented by the following formula (O), (P) or (Q):

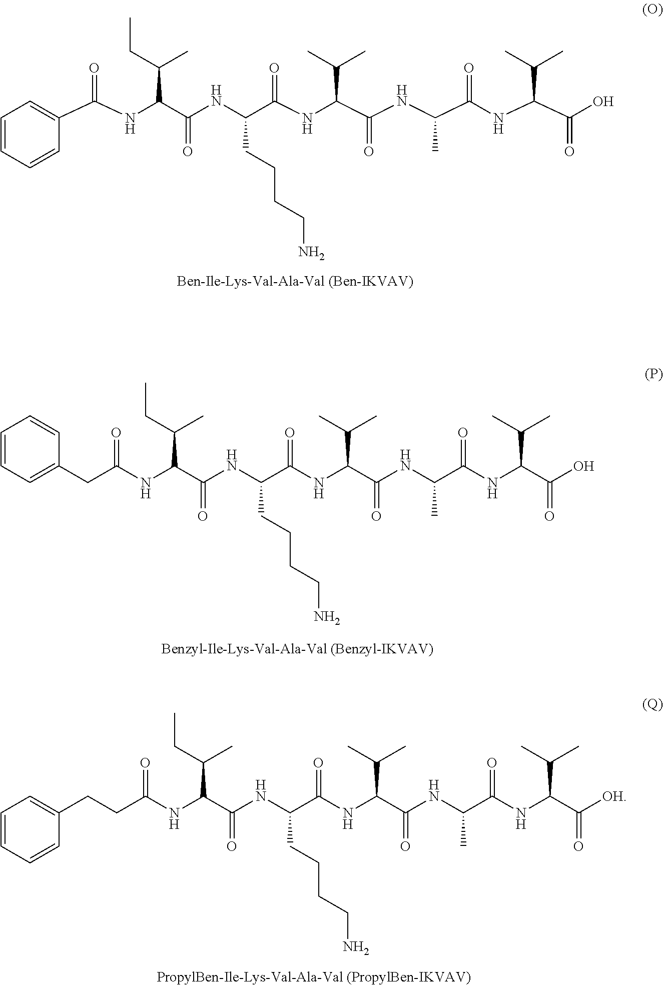 [SMILES: CC[C@@H](C)C(NC(=O)c1ccccc1)C(=O)N[C@@H](CCCCN)C(=O)N[C@H](C(=O)N[C@@H](C)C(=O)N[C@H](C(=O)O)C(C)C)C(C)C ; CC[C@@H](C)C(NC(=O)Cc1ccccc1)C(=O)N[C@@H](CCCCN)C(=O)N[C@H](C(=O)N[C@@H](C)C(=O)N[C@H](C(=O)O)C(C)C)C(C)C ; CC[C@@H](C)C(NC(=O)CCc1ccccc1)C(=O)N[C@@H](CCCCN)C(=O)N[C@H](C(=O)N[C@@H](C)C(=O)N[C@H](C(=O)O)C(C)C)C(C)C]

In one embodiment, A is phenyl substituted by fluorine; R₁ and R₂ are hydrogen atoms; R₃ and R₄ are independently selected from the group consisting of a hydrogen atom, C1-C10 alkyl, C7-C10 aralkyl and C1-C10 aminoalkyl; x is 1; and y is 6.

For example, the peptide molecular material of the present invention has a structure represented by the following formula (R):

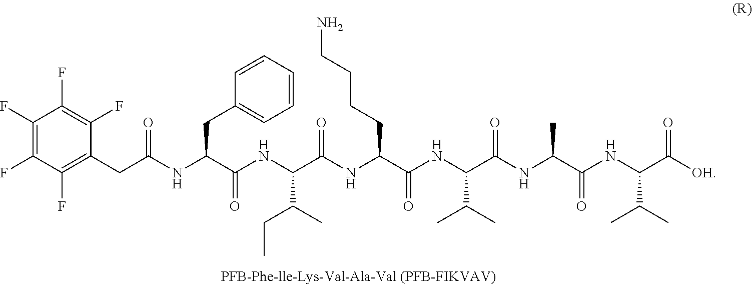 [SMILES: CCC(C)[C@H](NC(=O)[C@H](Cc1ccccc1)NC(=O)Cc1c(F)c(F)c(F)c(F)c1F)C(=O)N[C@@H](CCCCN)C(=O)N[C@H](C(=O)N[C@@H](C)C(=O)N[C@H](C(=O)O)C(C)C)C(C)C]

The present invention further provides a self-assembled hydrogel comprising the peptide molecular material of the present invention.

The peptide molecular material of the present invention is prepared by designing halogen-substituted or unsubstituted aryl at N-terminal of the peptide sequence, so that the peptide molecular material of the present invention is capable of self-assembling to a hydrogel without the self-assembled group Fmoc. Further, dimethyl sulfoxide (DMSO), which has cytotoxicity, does not need to be added for improving the stability of the hydrogel. In addition, the self-assembled hydrogel of the present invention has good stability obtained by properly adjusting the amino acid stably in a physiological condition (pH=7.4) and a human body temperature (the hydrogel of the present invention has storage modulus of >10000 Pa) and has low cytotoxicity, so that it has the advantages of promoting cell adhesion and relatively lower cost.

### BRIEF DESCRIPTION OF THE DRAWINGS

FIG. 1 shows the optical images of hydrogels (A) PFB-F (1 wt %, pH=5), (B) PFB-FG (1 wt %, pH=6-7), (C) PFB-YF (1 wt %, pH=7), (D) PFB-FF (1 wt %, pH=9-10), (E) PFB-FA (1 wt %, pH=6-7), (F) PFB-FV (1 wt %, pH=6-7), (G) PFB-VF (1 wt %, pH=7), (H) PFB-IF (pH=7), (I) PFB-LF (pH=7), (J) PFB-_{D}-L-_{D}-F (1 wt %, pH=7-8), (K) 4-MFB-FF (2 wt %, pH=7-8), (L) PFB-GFF (1 wt %, pH=5-6), (M) PFB-IKVAV (1 wt %, pH=7-8), (N) 4-MFB-IKVAV (1 wt %, pH=9), (O) Ben-IKVAV (1 wt %, pH=2-4), (P) Benzyl-IKVAV (1 wt %, pH=4), (Q) PropylBen-IKVAV, and (R) PFB-FIKVAV (1 wt %, pH=5-8).

FIG. 2A shows the transmission electron microscopy images of hydrogels (A) to (M), (P) and (R) at 37° C.

FIG. 2B shows the transmission electron microscopy images of hydrogels (I) and (J) at 4° C.

FIG. 3 shows the relationship between the storage modulus and the loss modulus of hydrogels (A) to (R).

FIG. 4A shows the Hela cell viability assays of hydrogels (A), (B) and (C).

FIG. 4B shows the CTX TNA2 cell viability assays of hydrogels (A), (B), (C) and (I).

FIG. 4C shows the MCF-7 cell viability assay of hydrogel (C).

FIG. 4D shows the CTX cell viability assay of hydrogel (G), (H), (I) and (J).

FIG. 4E shows the PC3 cell viability assay of hydrogel (H) and (K).

FIG. 4F shows the WS1 cell viability assay of hydrogel (C).

FIG. 4G shows the 3A6 cell viability assay of hydrogel (C).

FIG. 5A shows the optical images obtained from the wound healing assays of hydrogels (A), (B), (C), (E), (F) and Control (no compound added) (the tested cell: Hela cell).

FIG. 5B shows the optical images obtained from the wound healing assays of hydrogel (C) and Control (the tested cell: CTX TNA2 cell).

FIG. 5C shows the optical images obtained from the wound healing assays of hydrogel (G), (H) and Control (the tested cell: PC3 cell).

FIG. 5D shows the optical images obtained from the wound healing assays of hydrogel (C) and Control (the tested cell: 3A6 cell).

FIG. 6 shows the drug release assay of hydrogel (C) containing the anticancer drug, doxorubicin (DOX).

FIG. 7A shows the result of 3D cell culture of hydrogel (C) (the tested cell: CTX TNA2 cell).

FIG. 7B shows the result of 3D cell culture of hydrogel (H) (the tested cell: CTX).

FIG. 7C shows the result of 3D cell culture of hydrogel (H) (the tested cell: 3A6).

### DETAILED DESCRIPTION OF THE INVENTION

The following specific examples are used for illustrating the present invention. A person skilled in the art can easily conceive the other advantages and effects of the present invention. The present invention can also be implemented by different specific cases be enacted or application, the details of the instructions can also be based on different perspectives and applications in various modifications and changes do not depart from the spirit of the creation.

The present invention provides a novel peptide molecular material that is prepared by an organic synthesis method, i.e., by combining halogen-substituted aryl and a peptide molecular.

In the present invention, a peptide derivative is prepared by a solid phase peptide synthesis (SPPS) method. In this method, one or more peptides are combined together in the manner of chemical binding, and then the N-terminal of the combined peptide is linked to halogen-substituted or unsubstitutedphenyl.

The following examples are used to illustrate the present invention. The examples below should not be taken as a limitation to the scope of the invention.

### Material, Technique and General Process

The material, technique and general process in the present invention are suitable for the following examples. All used chemical reagents and solutions can be obtained from suppliers. ¹H and ¹³C spectrums were measured by Bruker DRX-300. LC-MS was measured by MICROMASS Q-Tof. TEM was measured by Hitachi HT7700 Bio-transmission electron microscope.

In the present invention, Rheological test was performed by Anton Paar rheometer. MTT cell viability assay was performed by Sunrise absorbance microplate reader (DV990/BV4 GDV Programmable MPT reader).

### Example 1

### Peptide Molecular Material Synthesis

### Example 1-A

### PFB-Phe (PFB-F) Synthesis

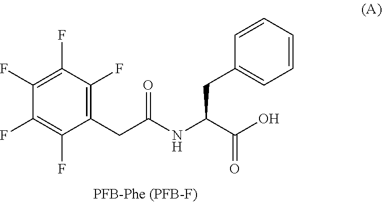 [SMILES: O=C(Cc1c(F)c(F)c(F)c(F)c1F)N[C@@H](Cc1ccccc1)C(=O)O]

A peptide derivative was prepared by using solid phase peptide synthesis (SPPS) of 2-chlorotrityl chloride resin (100 to 200 mesh and 0.3 to 0.8 mmol/g), the corresponding Fmoc-L-phenylalanine and pentafluorophenyl acetic acid. First, 2.4 g of resin was swelled in anhydrous dichloromethane (DCM) for 30 minutes. The resin in anhydrous N,N-dimethylformamide (DMF) and N,N-diisopropylethylamine (DIEA) (1.3 mL, 7.5 mmol) was then loaded with Fmoc-L-phenylalanine (1.16 g, 3 mmol) for 1 hour. During the deprotection of Fmoc groups, 20% of piperidine in a DMF solution was used for 20 minutes, and then repeated twice (2 minutes for each time). Subsequently, pentafluorophenyl acetic acid (0.68 g, 3 mmol) was coupled to free amino by using O-(benzotriazol-1-yl)-N,N,N′,N′-tetramethyluroniumhexafluorophosphate (HBTU) (1.14 g, 3 mmol) and DIEA (1.3 mL, 7.5 mmol) as coupling agents. After that, the reaction mixture was stirred overnight and the excess reagent was removed by DMF, DCM, methanol (MeOH) and hexane. The peptide derivative was cut off for 3 hours by using a 90% solution of trifluoroacetic acid in deionized water. The obtained solution was dried with air and was precipitated by adding ether to obtain the target product. The solid was vacuum dried to remove the remaining solution (white solid: 0.24 g). ¹H NMR (300 MHz, DMSO-d6): δ=2.85-3.00 (m, 1H), 3.00-3.20 (m, 1H), 3.65 (s, 2H), 4.46 (m, 1H), 7.20-7.40 (m, 5H), 8.62 (d, J=9.0 Hz, 1H); ¹³C NMR (125 MHz, DMSO-d6): δ=28.5, 36.8, 53.9, 110.2, 126.4, 128.1, 129.1, 136.7, 137.7, 143.8, 144.8, 166.6, 172.7; MS (ESI⁻): calculated 373.07; measured (M-H)⁻=372.00.

### Example 1-B

### PFB-Phe-Gly (PFB-FG) Synthesis

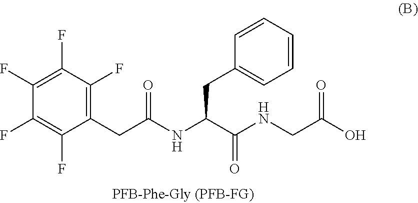 [SMILES: O=C(O)CNC(=O)[C@H](Cc1ccccc1)NC(=O)Cc1c(F)c(F)c(F)c(F)c1F]

A peptide derivative was prepared by using solid phase peptide synthesis (SPPS) of 2-chlorotrityl chloride resin (100 to 200 mesh and 0.3 to 0.8 mmol/g), the corresponding Fmoc-glycine, Fmoc-L-phenylalanine and pentafluorophenyl acetic acid. First, 1.2 g of resin was swelled in anhydrous DCM for 30 minutes. The resin in anhydrous DMF and DIEA (0.83 mL, 5 mmol) was then loaded with Fmoc-glycine (0.6 g, 2 mmol) for 1 hour. During the deprotection of Fmoc groups, 20% of piperidine in a DMF solution was used for 20 minutes, and then repeated twice (2 minutes for each time). Subsequently, Fmoc-L-phenylalanine (0.775 g, 2 mmol) was coupled to free amino for 30 minutes by using HBTU (0.758 g, 2 mmol) and DIEA (0.83 mL, 5 mmol) as coupling agents. After that, during the deprotection of Fmoc groups, 20% of piperidine in a DMF solution was used for 20 minutes, and then repeated twice (2 minutes for each time). Finally, pentafluorophenyl acetic acid (0.45 g, 2 mmol) was coupled to free amino by using HBTU (0.758 g, 2 mmol) and DIEA (0.83 mL, 5 mmol) as coupling agents. The reaction mixture was stirred overnight and the excess reagent was removed by DMF, DCM, MeOH and hexane. The peptide derivative was cut off for 3 hours by using a 90% solution of trifluoroacetic acid in deionized water. The obtained solution was dried with air and was precipitated by adding ether to obtain the target product. The solid was vacuum dried to remove the remaining solution (white solid: 0.21 g). ¹H NMR (300 MHz, DMSO-d6): δ=2.70-2.85 (m, 1H), 3.05-3.15 (m, 1H), 3.63 (d, J=9.3 Hz, 2H), 3.82 (d, J=5.7 Hz, 2H), 4.55-4.65 (m, 1H), 7.20-7.35 (m, 5H), 8.46 (t, J=5.7 Hz, 1H), 8.56 (d, J=8.4 Hz, 1H); ¹³C NMR (75 MHz, DMSO-d6): δ=29.1, 38.2, 41.2, 54.3, 54.4, 110.7, 127.5, 128.9, 130.0, 137.2, 138.2, 145.3, 166.9, 171.6, 171.7; MS (ESI⁻): calculated 430.10; measured 429.10.

### Example 1-C

### PFB-Tyr-Phe (PFB-YF) Synthesis

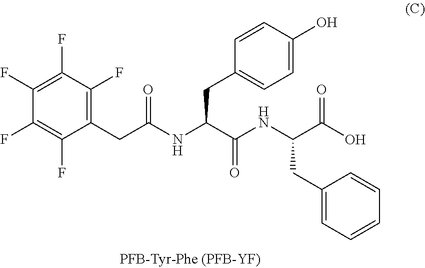 [SMILES: O=C(Cc1c(F)c(F)c(F)c(F)c1F)N[C@@H](Cc1ccc(O)cc1)C(=O)N[C@@H](Cc1ccccc1)C(=O)O]

A peptide derivative was prepared by using solid phase peptide synthesis (SPPS) of 2-chlorotrityl chloride resin (100 to 200 mesh and 0.3 to 0.8 mmol/g), the corresponding Fmoc-L-phenylalanine, O-tert-butyl-L-tyrosine and pentafluorophenyl acetic acid. First, 1.2 g of resin was swelled in anhydrous DCM for 30 minutes. The resin in anhydrous DMF and DIEA (0.8 mL, 5 mmol) was loaded with Fmoc-L-phenylalanine (0.78 g, 2 mmol) for 1 hour. During the deprotection of Fmoc groups, 20% of piperidine in a DMF solution was used for 20 minutes, and then repeated twice (2 minutes for each time). Subsequently, O-tert-butyl-L-tyrosine (2.3 g, 5 mmol) was coupled to free amino for 30 minutes by using HBTU (0.76 g, 2 mmol) and DIEA (2.1 mL, 12.5 mmol) as coupling agents. After that, during the deprotection of Fmoc groups, 20% of piperidine in a DMF solution was used for 20 minutes, and then repeated twice (2 minutes for each time). Finally, pentafluorophenyl acetic acid (0.45 g, 2 mmol) was coupled to free amino by using HBTU (0.76 g, 2 mmol) and DIEA (0.83 mL, 5 mmol) as coupling agents. The reaction mixture was stirred overnight and the excess reagent was removed by DMF, DCM, MeOH and hexane. The peptide derivative was cut off for 3 hours by using a 90% solution of trifluoroacetic acid in deionized water. The obtained solution was dried with air and was precipitated by ether to obtain the target product. The solid was vacuum dried to remove the remaining solution (white solid: 0.33 g). ¹H NMR (300 MHz, DMSO-d6): δ=2.60-2.70 (m, 1H), 1.85-3.20 (m, 3H), 3.59 (s, 2H), 4.40-4.55 (m, 2H), 6.65 (d, J=9.0 Hz, 2H), 7.04 (d, J=9.0 Hz, 2H), 7.20-7.35 (m, 5H), 8.4 (d, J=9.0 Hz, 2H); ¹³C NMR (75 MHz, DMSO-d6): δ=28.6, 36.6, 36.9, 53.5, 54.1, 110.4, 114.7, 126.4, 127.6, 128.1, 129.1, 130.0, 136.7, 137.4, 139.3, 144.8, 155.7, 166.2, 171.1, 172.7; MS (ESI⁻): calculated 536.45; measured 535.1.

### Example 1-D

### PFB-Phe-Phe (PFB-FF) Synthesis

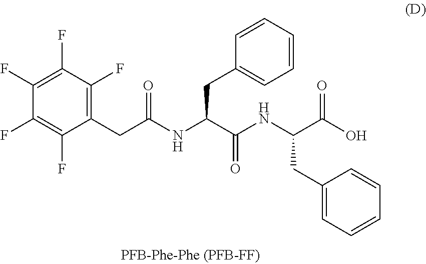 [SMILES: O=C(Cc1c(F)c(F)c(F)c(F)c1F)N[C@@H](Cc1ccccc1)C(=O)N[C@@H](Cc1ccccc1)C(=O)O]

A peptide derivative was prepared by using solid phase peptide synthesis (SPPS) of 2-chlorotrityl chloride resin (100 to 200 mesh and 0.3 to 0.8 mmol/g), which was treated with Fmoc-L-phenylalanine twice, and pentafluorophenyl acetic acid. First, 2.4 g of resin was swelled in anhydrous DCM for 30 minutes. Then, the resin in anhydrous DMF and DIEA (1.3 mL, 7.5 mmol) was loaded with Fmoc-L-phenylalanine (1.16 g, 3 mmol) for 1 hour. During the deprotection of Fmoc groups, 20% of piperidine in a DMF solution was used for 20 minutes, and then repeated twice (2 minutes for each time). Subsequently, Fmoc-L-phenylalanine (1.55 g, 4 mmol) was coupled to free amino for 30 minutes by using HBTU (1.52 g, 4 mmol) and DIEA (1.7 mL, 10.0 mmol) as coupling agents. After that, during the deprotection of Fmoc groups, 20% of piperidine in a DMF solution was used for 20 minutes, and then repeated twice (2 minutes for each time). Finally, pentafluorophenyl acetic acid (1.356 g, 6 mmol) was coupled to free amino by using HBTU (2.28 g, 6 mmol) and DIEA (2.5 mL, 15.0 mmol) as coupling agents. The reaction mixture was stirred overnight and the excess reagent was removed by DMF, DCM, MeOH and hexane. The peptide derivative was cut off for 3 hours by using a 90% solution of trifluoroacetic acid in deionized water. The obtained solution was dried with air and was precipitated by adding ether to obtain the target product. The solid was vacuum dried to remove the remaining solution (white solid: 0.56 g). ¹H NMR (300 MHz, DMSO-d6): δ=2.70-2.80 (m, 1H), 2.90-3.15 (m, 3H), 3.58 (s, 2H), 4.45-4.55 (m, 1H), 4.55-4.65 (m, 1H), 7.20-7.35 (m, 10H), 8.35-8.50 (m, 2H); ¹³C NMR (125 MHz, DMSO-d6): δ=28.6, 36.8, 37.9, 53.6, 53.8, 110.3, 126.2, 126.4, 127.9, 128.1, 129.1, 129.2, 137.5, 137.6, 139.2, 144.8, 166.3, 170.9, 172.7; MS (ESI⁻): calculated 520.14; measured 519.20.

### Example 1-E

### PFB-Phe-Ala (PFB-FA) Synthesis

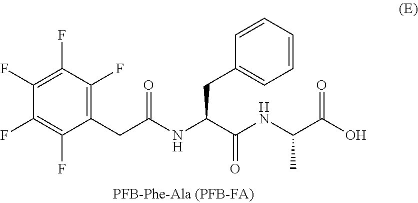 [SMILES: C[C@H](NC(=O)[C@H](Cc1ccccc1)NC(=O)Cc1c(F)c(F)c(F)c(F)c1F)C(=O)O]

A peptide derivative was prepared by using solid phase peptide synthesis (SPPS) of 2-chlorotrityl chloride resin (100 to 200 mesh and 0.3 to 0.8 mmol/g), the corresponding Fmoc-L-alanine, Fmoc-L-phenylalanine and pentafluorophenyl acetic acid. First, 1.2 g of resin was swelled in anhydrous DCM for 30 minutes. Then, the resin in anhydrous DMF and DIEA (0.83 mL, 5 mmol) was loaded with Fmoc-L-alanine (0.62 g, 2 mmol) for 1 hour. During the deprotection of Fmoc groups, 20% of piperidine in a DMF solution was used for 20 minutes, and then repeated twice (2 minutes for each time). Subsequently, Fmoc-L-phenylalanine (0.77 g, 2 mmol) was coupled to free amino for 30 minutes by using HBTU (0.94 g, 2 mmol) and DIEA (0.83 mL, 5 mmol) as coupling agents. After that, during the deprotection of Fmoc groups, 20% of piperidine in a DMF solution was used for 20 minutes, and then repeated twice (2 minutes for each time). Finally, pentafluorophenyl acetic acid (0.67 g, 3 mmol) was coupled to free amino by using HBTU (1.13 g, 3 mmol) and DIEA (1.3 mL, 7.5 mmol) as coupling agents. The reaction mixture was stirred overnight and the excess reagent was removed by DMF, DCM, MeOH and hexane. The peptide derivative was cut off for 3 hours by using a 90% solution of trifluoroacetic acid in deionized water. The obtained solution was dried with air and was precipitated by adding ether to obtain the target product. The solid was vacuum dried to remove the remaining solution (white solid: 0.29 g). ¹H NMR (300 MHz, DMSO-d6): δ=1.34 (d, J=7.2 Hz, 3H), 2.70-2.85 (m, 2H), 3.05-3.15 (m, 2H), 3.61 (d, J=4.8 Hz, 2H), 4.20-4.35 (m, 1H), 4.55-4.65 (m, 1H), 7.20-7.35 (m, 5H), 8.45 (d, J=7.5 Hz, 1H), 8.52 (d, J=8.7 Hz, 1H); ¹³C NMR (75 MHz, DMSO-d6): δ=18.0, 29.5, 38.7, 48.5, 54.8, 111.2, 127.2, 128.9, 130.1, 137.6, 138.7, 145.7, 167.4, 171.8, 174.9; MS (ESI⁻): calculated 444.35; measured 443.0.

### Example 1-F

### PFB-Phe-Val (PFB-FV) Synthesis

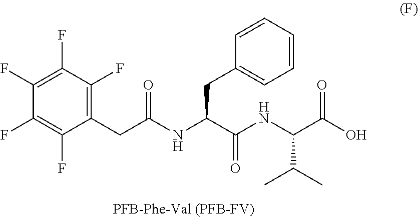 [SMILES: CC(C)[C@H](NC(=O)[C@H](Cc1ccccc1)NC(=O)Cc1c(F)c(F)c(F)c(F)c1F)C(=O)O]

A peptide derivative was prepared by using solid phase peptide synthesis (SPPS) of 2-chlorotrityl chloride resin (100 to 200 mesh and 0.3 to 0.8 mmol/g), the corresponding Fmoc-L-valine, Fmoc-L-phenylalanine and pentafluorophenyl acetic acid. First, 1.2 g of resin was swelled in anhydrous DCM for 30 minutes. Then, the resin in anhydrous DMF and DIEA (0.83 mL, 5 mmol) was loaded with Fmoc-L-valine (0.68 g, 2 mmol) for 1 hour. During the deprotection of Fmoc groups, 20% of piperidine in a DMF solution was used for 20 minutes, and then repeated twice (2 minutes for each time). Subsequently, Fmoc-L-phenylalanine (0.78 g, 2 mmol) was coupled to free amino for 30 minutes by using HBTU (0.76 g, 2 mmol) and DIEA (0.83 mL, 5 mmol) as coupling agents. After that, during the deprotection of Fmoc groups, 20% of piperidine in a DMF solution was used for 20 minutes, and then repeated twice (2 minutes for each time). Finally, pentafluorophenyl acetic acid (0.45 g, 3 mmol) was coupled to free amino by using HBTU (1.14 g, 3 mmol) and DIEA (1.25 mL, 7.5 mmol) as coupling agents. The reaction mixture was stirred overnight and the excess reagent was removed by DMF, DCM, MeOH and hexane. The peptide derivative was cut off for 3 hours by using a 90% solution of trifluoroacetic acid in deionized water. The obtained solution was dried with air and was precipitated by adding ether to obtain the target product. The solid was vacuum dried to remove the remaining solution (white solid: 0.48 g). ¹H NMR (300 MHz, DMSO-d6): δ=0.92 (d, J=6.9 Hz, 6H), 2.05-2.20 (m, 1H), 2.75-2.90 (m, 1H), 3.05-3.15 (m, 1H), 3.61 (s, 2H), 4.20 (dd, J=5.8, 8.6 Hz, 1H), 4.65-4.75 (m, 1H), 7.20-7.35 (m, 5H), 8.17 (d, J=8.1 Hz, 1H), 8.52 (d, J=8.4 Hz, 1H); ¹³C NMR (75 MHz, DMSO-d6): δ=18.9, 20.0, 29.6, 38.5, 54.8, 58.2, 111.2, 127.2, 128.9, 130.2, 137.7, 138.6, 141.9, 145.8, 167.5, 172.2, 173.7; MS (ESI⁻): calculated 472.41; measured 471.1.

### Example 1-G

### PFB-Val-Phe (PFB-VF) Synthesis

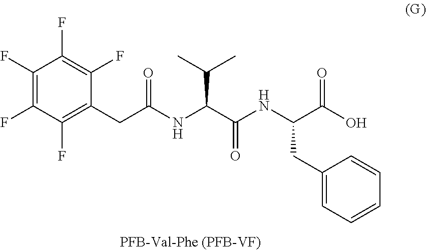 [SMILES: CC(C)[C@H](NC(=O)Cc1c(F)c(F)c(F)c(F)c1F)C(=O)N[C@@H](Cc1ccccc1)C(=O)O]

A peptide derivative was prepared by using solid phase peptide synthesis (SPPS) of 2-chlorotrityl chloride resin (100 to 200 mesh and 0.3 to 0.8 mmol/g), the corresponding Fmoc-L-valine, Fmoc-L-phenylalanine and pentafluorophenyl acetic acid. First, 1.2 g of resin was swelled in anhydrous DCM for 30 minutes. Then, the resin in anhydrous DMF and DIEA (0.83 mL, 5 mmol) was loaded with Fmoc-L-phenylalanine (0.77 g, 2 mmol) for 1 hour. During the deprotection of Fmoc groups, 20% of piperidine in a DMF solution was used for 20 minutes, and then repeated twice (2 minutes for each time). Subsequently, Fmoc-L-valine (0.68 g, 2 mmol) was coupled to free amino for 30 minutes by using HBTU (0.76 g, 2 mmol) and DIEA (0.83 mL, 5 mmol) as coupling agents. After that, during the deprotection of Fmoc group, 20% of piperidine in a DMF solution was used for 20 minutes, and then repeated twice (2 minutes for each time). Finally, pentafluorophenyl acetic acid (1.13 g, 5 mmol) was coupled to free amino by using HBTU (1.9 g, 5 mmol) and DIEA (20.8 mL, 12.5 mmol) as coupling agents. The reaction mixture was stirred overnight and the excess reagent was removed by DMF, DCM, MeOH and hexane. The peptide derivative was cut off for 3 hours by using a 90% solution of trifluoroacetic acid in deionized water. The obtained solution was dried with air and was precipitated by adding ether to obtain the target product. The solid was vacuum dried to remove the remaining solution (white solid: 0.33 g). ¹H NMR (300 MHz, [D₆]DMSO, 25° C.): δ=0.8-0.95 (m, 6H; CH₃), 1.90-2.205 (m, 1H; CH), 2.85-3.00 (m, 1H; CH₂), 3.05-3.15 (m, 1H; CH₂), 3.60-3.85 (m, 2H; CH₂), 4.20-4.30 (m, 1H; CH), 4.40-4.50 (m, 1H; CH), 7.15-7.35 (m, 5H; CH), 8.25-8.40 (m, 2H; NH); ¹³C NMR (125 MHz, [D₆]DMSO, 25° C.): δ=17.9, 19.1, 28.5, 30.9, 36.6, 53.4, 57.5, 110.7, 126.4, 128.1, 129.1, 136.8, 137.7, 139.2, 144.9, 166.6, 170.8, 172.8; MS (ESI⁻): calculated 472.14; measured 471.3 [M-H]⁻.

### Example 1-H

### PFB-Ile-Phe (PFB-IF) Synthesis

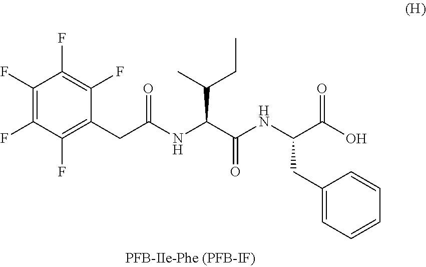 [SMILES: CCC(C)[C@H](NC(=O)Cc1c(F)c(F)c(F)c(F)c1F)C(=O)N[C@@H](Cc1ccccc1)C(=O)O]

A peptide derivative was prepared by using solid phase peptide synthesis (SPPS) of 2-chlorotrityl chloride resin (100 to 200 mesh and 0.3 to 0.8 mmol/g), the corresponding Fmoc-L-phenylalanine, Fmoc-L-isoleucine and pentafluorophenyl acetic acid. First, 1.2 g of resin was swelled in anhydrous DCM for 30 minutes. Then, the C-terminal of the resin in anhydrous DMF and DIEA (0.83 mL, 5 mmol) was loaded with Fmoc-L-phenylalanine (0.78 g, 2 mmol). During the deprotection of Fmoc groups, 20% of piperidine in a DMF solution was used for 30 minutes, and then repeated twice (2 minutes for each time). Subsequently, Fmoc-L-isoleucine (0.71 g, 2 mmol) was coupled to free amino for 40 minutes by using HBTU (0.76 g, 2 mmol) and DIEA (0.83 mL, 5 mmol) as coupling agents. After that, during the deprotection of Fmoc groups, 20% of piperidine in a DMF solution was used for 30 minutes, and then repeated twice (2 minutes for each time). Finally, pentafluorophenyl acetic acid (0.68 g, 3 mmol) was coupled to free amino by using HBTU (1.14 g, 3 mmol) and DIEA (1.25 mL, 5 mmol) as coupling agents. The reaction mixture was stirred overnight and the excess reagent was removed by DMF, DCM, MeOH and hexane. The peptide derivative was cut off for 3 hours by using a 90% solution of trifluoroacetic acid in deionized water. The obtained solution was dried with air and was precipitated by adding ether to obtain the target product. The solid was vacuum dried to remove the remaining solution (white solid: 0.16 g). ¹H NMR (300 MHz, [D₆]DMSO, 25° C.): δ=0.75-0.95 (m, 6H; CH₃), 1.00-1.18 (m, 1H; CH₂), 1.37-1.53 (m, 1H; CH₂), 1.68-1.85 (m, 1H; CH), 2.88-3.15 (m, 2H; CH₂), 3.59-3.81 (m, 2H; CH₂), 4.28 (t, J=8.1 Hz, 1H; CH), 4.41-4.53 (m, 1H; CH), 7.17-7.35 (m, 5H; CH), 8.27-8.43 (m, 2H; NH); ¹³C NMR (75 MHz, [D₆]DMSO, 25° C.): δ=11.9, 16.1, 24.9, 29.5, 37.5, 37.9, 54.3, 57.7, 111.6, 127.3, 129.0, 130.0, 137.6, 138.5, 145.7, 167.4, 171.8, 173.7; MS (ESI⁻): calculated 486.16; measured 485.40 [M-H]⁻.

### Example 1-I

### PFB-Leu-Phe (PFB-LF) Synthesis

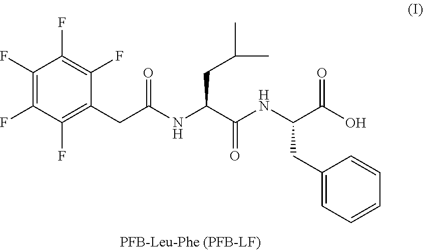 [SMILES: CC(C)C[C@H](NC(=O)Cc1c(F)c(F)c(F)c(F)c1F)C(=O)N[C@@H](Cc1ccccc1)C(=O)O]

A peptide derivative was prepared by using solid phase peptide synthesis (SPPS) of 2-chlorotrityl chloride resin (100 to 200 mesh and 0.3 to 0.8 mmol/g), the corresponding Fmoc-L-phenylalanine, Fmoc-L-leucine and pentafluorophenyl acetic acid. First, 1.2 g of resin was swelled in anhydrous DCM for 30 minutes. Then, the resin in anhydrous DMF and DIEA (0.83 mL, 5 mmol) was loaded with Fmoc-L-phenylalanine (0.78 g, 2 mmol) for 1 hour. During the deprotection of Fmoc groups, 20% of piperidine in a DMF solution was used for 30 minutes, and then repeated twice (2 minutes for each time). Subsequently, Fmoc-L-leucine (0.71 g, 2 mmol) was coupled to free amino for 40 minutes by using HBTU (0.76 g, 2 mmol) and DIEA (0.83 mL, 5 mmol) as coupling agents. After that, during the deprotection of Fmoc groups, 20% of piperidine in a DMF solution was used for 30 minutes, and then repeated twice (2 minutes for each time). Finally, pentafluorophenyl acetic acid (0.68 g, 3 mmol) was coupled to free amino by using HBTU (1.13 g, 3 mmol) and DIEA (1.3 mL, 7.5 mmol) as coupling agents. The reaction mixture was stirred overnight and the excess reagent was removed by DMF, DCM, MeOH and hexane. The peptide derivative was cut off for 3 hours by using a 90% solution of trifluoroacetic acid in deionized water. The obtained solution was dried with air and was precipitated by adding ether to obtain the target product. The solid was vacuum dried to remove the remaining solution (white solid: 0.34 g). ¹H NMR (300 MHz, [D₆]DMSO, 25° C.): δ=0.81-1.00 (m, 6H; CH₃), 1.40-1.55 (t, 2H; CH₂), 1.55-1.70 (m, 1H; CH), 2.85-3.15 (m, 2H; CH₂), 3.68 (s, 2H; CH₂), 4.35-4.50 (m, 2H; CH), 7.15-7.35 (m, 5H; CH), 8.25 (d, J=8.1 Hz, 1H; NH), 8.41 (d, J=8.4 Hz, 1H; NH); ¹³C NMR (125 MHz, [D₆]DMSO, 25° C.): δ=22.6, 23.9, 25.1, 29.5, 37.4, 51.9, 54.3, 111.4, 127.3, 129.0, 130.0, 137.7, 138.5, 145.8, 167.3, 172.7, 173.7; MS (ESI⁻): m/z (%): calculated 486.16; measured 485.30 [M-H]⁻.

### Example 1-J

### PFB-_{D}-L-_{D}-F (PFB-_{D}-L-_{D}-F) Synthesis

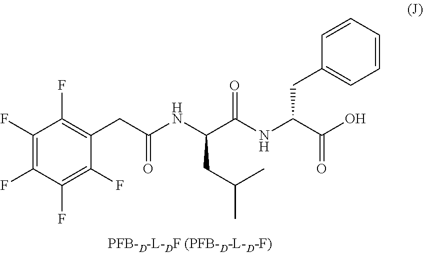 [SMILES: CC(C)C[C@@H](NC(=O)Cc1c(F)c(F)c(F)c(F)c1F)C(=O)N[C@H](Cc1ccccc1)C(=O)O]

A peptide derivative was prepared by using solid phase peptide synthesis (SPPS) of 2-chlorotrityl chloride resin (100 to 200 mesh and 0.3 to 0.8 mmol/g) and the corresponding Fmoc-D-phenylalanine, Fmoc-D-leucine and pentafluorophenyleacetic acid. First, 1.2 g of resin was swelled in anhydrous DCM for 30 minutes. Then, Fmoc-D-phenylalanine (0.78 g, 2 mmol) was loaded on the resin in anhydrous N,N′-dimethylformamide (DMF) and N,N-Diisopropylethylamine (DIEA) (0.83 mL, 5 mmol) for 1 hour. 20% piperidine in DMF was used during the deprotection of Fmoc group for 30 minutes and then repeated twice (2 minutes for each time). Then, the Fmoc-D-leucine (0.71 g, 2 mmol) was coupled to the free amino group using O-(Benzotriazol-1-yl)-N,N,N′,N′-tetramethyluraniumhexafluorophosphate (HBTU) (0.76 g, 2 mmol) and N,N-Diisopropylethylamine (DIEA) (0.83 mL, 5 mmol) as the coupling reagents for 40 minutes. Next, 20% piperidine in DMF was used during the deprotection of Fmoc group for 30 minutes and then repeated twice (2 minutes for each time). Finally, the pentafluoro benzeneacetic acid (0.68 g, 3 mmol) was also coupled to the free amino group using O-(Benzotriazol-1-yl)-N,N,N′,N′-tetramethyluraniumhexa-fluorophosphate (HBTU) (1.13 g, 3 mmol) and N,N-Diisopropylethylamine (DIEA) (1.3 mL, 7.5 mmol) as the coupling reagents. After the reaction mixture was stirred overnight, excessive reagents were removed by DMF, DCM, MeOH, and Hexane. The peptide derivative was cleaved using 90% of trifluoroacetic acid in DI water for 3 hours. The resulting solution was air-dried, and then diethyl ether was added to precipitate the target product. The solid was dried under vacuum to remove remaining solvent (white solid: 0.341 g). ¹H NMR (300 MHz, [D₆]DMSO, 25° C.): δ=0.80-0.95 (m, 6H; CH₃), 1.38-1.50 (t, J=7.5 Hz, 2H; CH₂), 1.55-1.65 (m, 1H; CH), 2.85-3.10 (m, 2H; CH₂), 3.68 (s, 2H; CH₂), 4.30-4.45 (m, 2H; CH), 7.15-7.35 (m, 5H; CH), 8.28 (d, J=8.1 Hz, 1H; NH), 8.41 (d, J=8.7 Hz, 1H; NH); ¹³C NMR (75 MHz, [D₆]DMSO, 25° C.): δ=22.5, 23.9, 25.0, 29.5, 37.4, 41.9, 51.9, 54.3, 111.4, 127.3, 129.0, 130.0, 137.8, 145.8, 167.3, 172.6, 173.6; MS [ESI⁻]: m/z (%): calculated 486.16, observed 485.0 [M-H]⁻.

### Example 1-K

### 4-Fluorobenzyl-Phe-Phe (4-MFB-FF) Synthesis

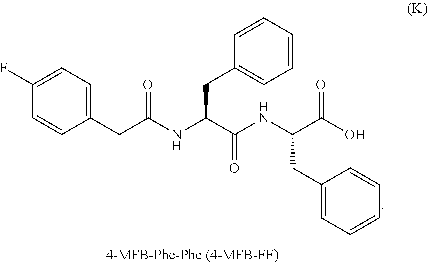 [SMILES: O=C(Cc1ccc(F)cc1)N[C@@H](Cc1ccccc1)C(=O)N[C@@H](Cc1ccccc1)C(=O)O]

A peptide/dye conjugate derivative of 4-MFB-FF was prepared by using SPPS of 2-chlorotrityl chloride resin, Fmoc-L-phenylalanine and 4-Fluorphenyl acetic acid. First, 1.2 g of resin was swelled in anhydrous DCM for 30 minutes. Then, Fmoc-L-phenylalanine (0.775 g, 2.000 mmol) was loaded on the resin in anhydrous N,N-dimethylformamide and N,N-diisopropylethylamine (DIEA; 0.830 mL, 5.000 mmol) for 1 hour. For the deprotection of the Fmoc group, piperidine (20% in DMF) was added and the sample was left for 20 minutes; this procedure was repeated twice (2 minutes for each time). Fmoc-L-phenylalanine (0.775 g, 2.000 mmol) was coupled to the free amino group using O-(benzotriazol-1-yl)-N,N,N′,N′-tetramethyluraniumhexafluorophosphate (HBTU) (0.758 g, 2.000 mmol) and N,N-diisopropylethylamine (DIEA) (0.830 mL, 5.000 mmol) as coupling agents for 30 minutes. Again, the sample was treated with piperidine (20% in DMF) for 20 minutes; this procedure was repeated twice (2 minutes for each time). Finally, 4-Fluorphenyl acetic acid (0.462 g, 3.000 mmol) was coupled to the free amino group using HBTU (1.137 g, 3.000 mmol) and DIEA (1.239 mL, 7.500 mmol) as coupling agents. After the reaction mixture had been stirred overnight, the peptide derivative was cleaved through treatment with CF₃CO₂H (90% in DI water) for 3 hours. The resulting solution was dried by air and then Et₂O was added to precipitate the target product. The solid was dried under vacuum to remove the remaining solvent (white solid: 0.307 g). ¹H NMR (300 MHz, [D₆]DMSO): δ=2.65-3.15 (m, 4H; CH₂), 3.55-3.65 (m, 2H; CH₂), 4.30-4.40 (m, 1H; CH), 4.45-4.60 (m, 1H; CH), 6.95-7.35 (m, 14H; CH), 8.10-8.20 (br, 1H; NH), 8.32 (d, J=9.00 Hz 1H; NH); ¹³C NMR (75 MHz, [D₆]DMSO): δ=37.7, 38.5, 42.1, 54.7, 58.5, 115.5, 115.8, 127.1, 127.2, 128.9, 129.0, 130.2, 131.6, 131.7, 133.3, 138.7, 138.8, 170.6, 172.0, 173.9; MS [ESI⁻]: calculated m/z 448.18, observed 447.2 [M-H]⁻.

### Example 1-L

### PFB-Gly-Phe-Phe (PFB-GFF) Synthesis

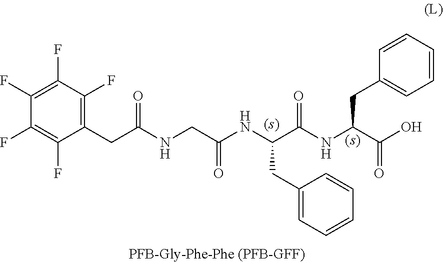 [SMILES: O=C(Cc1c(F)c(F)c(F)c(F)c1F)NCC(=O)N[C@@H](Cc1ccccc1)C(=O)N[C@@H](Cc1ccccc1)C(=O)O]

A peptide derivative was prepared by using solid phase peptide synthesis (SPPS) of 2-chlorotrityl chloride resin (100 to 200 mesh and 0.3 to 0.8 mmol/g), which was treated with Fmoc-L-phenylalanine twice, Fmoc-L-glycine and pentafluorophenyl acetic acid. First, 1.2 g of resin was swelled in anhydrous DCM for 30 minutes. Then, the resin in anhydrous DMF and DIEA (0.83 mL, 5 mmol) was loaded with Fmoc-L-phenylalanine (0.775 g, 2 mmol) for 1 hour. During the deprotection of Fmoc groups, 20% of piperidine in a DMF solution was used for 20 minutes, and then repeated twice (2 minutes for each time). Subsequently, Fmoc-L-phenylalanine (0.775 g, 2 mmol) was coupled to free amino for 30 minutes by using HBTU (0.758 g, 2 mmol) and DIEA (0.83 mL, 5 mmol) as coupling agents. Fmoc-glycine (0.6 g, 2 mmol) was coupled to free amino for 30 minutes by using HBTU (0.758 g, 2 mmol) and DIEA (0.83 mL, 5 mmol) as coupling agents. After that, during the deprotection of Fmoc groups, 20% of piperidine in a DMF solution was used for 20 minutes, and then repeated twice (2 minutes for each time). Finally, pentafluorophenyl acetic acid (0.45 g, 2 mmol) was coupled to free amino by using HBTU (0.758 g, 2 mmol) and DIEA (0.83 mL, 5 mmol) as coupling agents. The reaction mixture was stirred overnight and the excess reagent was removed by DMF, DCM, MeOH and hexane. The peptide derivative was cut off for 3 hours by using a 90% solution of trifluoroacetic acid in deionized water. The obtained solution was dried with air and was precipitated by adding ether to obtain the target product. The solid was vacuum dried to remove the remaining solution (white solid: 0.37 g). ¹H NMR (300 MHz, DMSO-d6): δ=2.70-2.80 (m, 1H), 2.90-3.15 (m, 3H), 3.55-3.90 (m, 4H), 4.40-4.50 (m, 1H), 4.55-4.65 (m, 1H), 7.15-7.35 (m, 10H), 8.14 (d, J=8.4 Hz, 1H), 8.35-8.45 (m, 2H); ¹³C NMR (75 MHz, DMSO-d6): δ=29.5, 37.6, 38.5, 43.0, 54.5, 111.2, 127.2, 127.4, 128.9, 129.2, 130.0, 130.1, 137.7, 138.4, 138.6, 145.8, 168.0, 169.1, 172.0, 173.6; MS (ESI⁻): calculated 577.50; measured 576.2.

### Example 1-M

### PFB-Ile-Lys-Val-Ala-Val (PFB-IKVAV) Synthesis

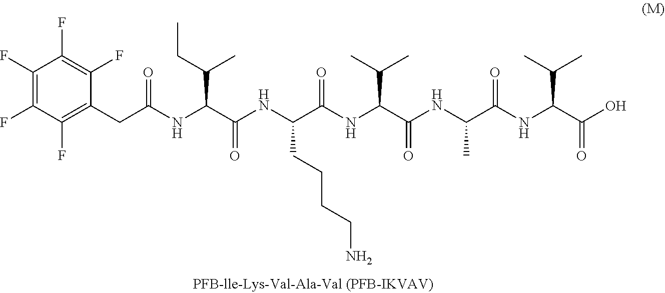 [SMILES: CCC(C)[C@H](NC(=O)Cc1c(F)c(F)c(F)c(F)c1F)C(=O)N[C@@H](CCCCN)C(=O)N[C@H](C(=O)N[C@@H](C)C(=O)N[C@H](C(=O)O)C(C)C)C(C)C]

A peptide derivative was prepared by using solid phase peptide synthesis (SPPS) of 2-chlorotrityl chloride resin (100 to 200 mesh and 0.3 to 0.8 mmol/g), which was treated with Fmoc-valine-OH, Fmoc-alanine-OH, Fmoc-valine-OH, Fmoc-lysine(Boc)-OH, Fmoc-isoleucine-OH and pentafluorophenyl acetic acid. First, 1.2 g of resin was swelled in anhydrous DCM for 30 minutes. Then, the resin in anhydrous DMF and DIEA (0.83 mL, mmol) was loaded with Fmoc-valine-OH (0.678 g, 2 mmol) for 1 hour. During the deprotection of Fmoc groups, 20% of piperidine in a DMF solution was used for 20 minutes, and then repeated twice (2 minutes for each time). Subsequently, Fmoc-alanine-OH (0.623 g, 2 mmol) was coupled to free amino for 40 minutes by using HBTU (0.758 g, 2 mmol) and DIEA (0.83 mL, 5 mmol) as coupling agents. After that, during the deprotection of Fmoc groups, 20% of piperidine in a DMF solution was used for 20 minutes, and then repeated twice (2 minutes for each time). Fmoc-valine-OH (0.678 g, 2 mmol) was coupled to free amino for 40 minutes by using HBTU (0.758 g, 2 mmol) and DIEA (0.83 mL, 5 mmol) as coupling agents. During the deprotection of Fmoc groups, 20% of piperidine in a DMF solution was used for 20 minutes, and then repeated twice (2 minutes for each time). After that, the above steps were repeated with Fmoc-lysine(Boc)-OH (0.937 g, 2 mmol) and Fmoc-isoleucine-OH (0.707 g, 2 mmol) for 40 minutes by using HBTU (0.758 g, 2 mmol) and DIEA (0.83 mL, 5 mmol) as coupling agents. Subsequently, during the deprotection of Fmoc groups, 20% of piperidine in a DMF solution was used for 20 minutes, and then repeated twice (2 minutes for each time). Finally, pentafluorophenyl acetic acid (0.79 g, 3.5 mmol) was coupled to free amino by using HBTU (1.3 g, 3.5 mmol) and DIEA (1.45 mL, 8.75 mmol) as coupling agents. The reaction mixture was stirred overnight and the excess reagent was removed by DMF, DCM, MeOH and hexane. The peptide derivative was cut off for 3 hours by using a 90% solution of trifluoroacetic acid in deionized water. The obtained solution was dried with air and was precipitated by adding ether to obtain the target product. The solid was vacuum dried to remove the remaining solution (white solid: 0.62 g). ¹H NMR (300 MHz, DMSO-d6): δ=0.8-2.2 (m, 32H), 2.77 (s, 2H), 3.75 (m, 2H), 4.10-4.50 (m, 5H), 7.65-7.80 (m, 4H), 7.91 (d, J=8.1 Hz, 1H), 8.06 (d, J=6.9 Hz, 1H), 8.19 (d, J=8.1 Hz, 1H), 8.42 (d, J=9.0 Hz, 1H); ¹³C NMR (75 MHz, DMSO-d6): δ=12.0, 16.3, 18.8, 19.1, 20.0, 20.1, 23.3, 25.2, 27.6, 29.6, 31.7, 32.1, 32.4, 37.3, 37.8, 48.9, 53.5, 58.0, 58.1, 58.3, 111.6, 117.8, 137.7, 145.9, 167.8, 171.3, 171.8, 172.3, 173.3, 173.8; MS (EST⁺): calculated 736.4; measured 737.4.

### Example 1-N

### 4-Fluorophenylacetic Acid-Ile-Lys-Val-Ala-Val (4-MFB-IKVAV) Synthesis

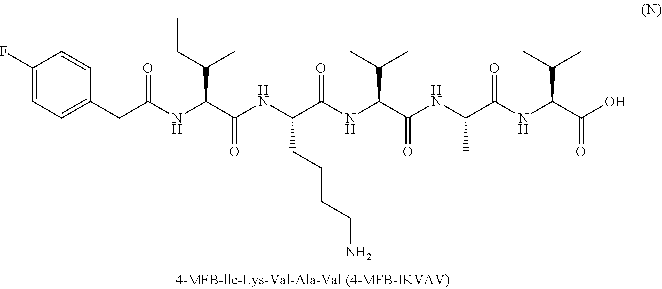 [SMILES: CCC(C)[C@H](NC(=O)Cc1ccc(F)cc1)C(=O)N[C@@H](CCCCN)C(=O)N[C@H](C(=O)N[C@@H](C)C(=O)N[C@H](C(=O)O)C(C)C)C(C)C]

A peptide derivative was prepared by using solid phase peptide synthesis (SPPS) of 2-chlorotrityl chloride resin (100 to 200 mesh and 0.3 to 0.8 mmol/g), the corresponding Fmoc-valine-OH, Fmoc-alanine-OH, Fmoc-valine-OH, Fmoc-lysine(Boc)-OH, Fmoc-isoleucine-OH and 4-fluorophenylacetic acid. First, the resin (0.4 g, 0.33 mmol) was swelled in anhydrous DCM for 30 minutes. The C-terminal of the resin in anhydrous DMF and DIEA (0.28 mL, 1.67 mmol) was loaded with Fmoc-valine-OH (0.23 g, 0.67 mmol) for 1 hour. During the deprotection of Fmoc groups, 20% of piperidine in a DMF solution was used for 20 minutes, and then repeated twice (2 minutes for each time). Subsequently, Fmoc-alanine-OH (0.21 g, 0.67 mmol) was coupled to free amino for 40 minutes by using HBTU (0.25 g, 0.67 mmol) and DIEA (0.28 mL, 1.67 mmol) as coupling agents. After that, during the deprotection of Fmoc groups, 20% of piperidine in a DMF solution was used for 20 minutes, and then repeated twice (2 minutes for each time). After that, Fmoc-valine-OH (0.23 g, 0.67 mmol) was coupled to free amino for 40 minutes by using HBTU (0.25 g, 0.67 mmol) and DIEA (0.28 mL, 1.67 mmol) as coupling agents. During the deprotection of Fmoc groups, 20% of piperidine in a DMF solution was used for 20 minutes, and then repeated twice (2 minutes for each time). Subsequently, as described in the above steps, Fmoc-lysine(Boc)-OH (0.31 g, 0.67 mmol) and Fmoc-isoleucine-OH (0.707 g, 2 mmol) were coupled to free amino for 40 minutes by using HBTU (0.25 g, 0.67 mmol) and DIEA (0.28 mL, 1.67 mmol) as coupling agents. After that, during the deprotection of Fmoc groups, 20% of piperidine in a DMF solution was used for 20 minutes, and then repeated twice (2 minutes for each time). Finally, 4-fluorophenylacetic acid (0.15 g, 1 mmol) was coupled to free amino by using HBTU (0.37 g, 1 mmol) and DIEA (0.42 mL, 2.5 mmol) as coupling agents. The reaction mixture was stirred overnight and the excess reagent was removed by DMF, DCM, MeOH and hexane. The peptide derivative was cut off for 3 hours by using a 90% solution of trifluoroacetic acid in deionized water.

The obtained solution was dried with air and was precipitated by adding ether to obtain the target product. The solid was vacuum dried to remove the remaining solution (white solid: 0.23 g). ¹H NMR (300 MHz, DMSO-d6): δ=0.75-2.15 (m, 32H), 2.77 (m, 2H), 3.3-3.7 (m, 2H), 4.15-4.50 (m, 5H, 7.10-7.80 (m, 7H), 7.92 (d, J=8.7 Hz, 1H), 8.08 (d, J=6.3 Hz, 1H), 8.16 (d, J=7.5 Hz, 1H), 8.22 (d, J=8.4 Hz, 1H); ¹³C NMR (75 MHz, DMSO-d6): δ=10.9, 15.3, 17.8, 17.8, 17.8, 18.1, 19.0, 19.1, 22.2, 24.2, 26.6, 30.0, 31.1, 36.6, 38.7, 41.0, 47.9, 52.4, 56.8, 57.0, 57.2, 114.8, 130.7, 130.8, 132.8, 170.0, 170.3, 171.1, 171.2, 172.2, 172.8; MS (EST⁺): calculated 664.4; measured 665.5 (M-H)⁺.

### Example 1-O

### Benzoic Acid-Ile-Lys-Val-Ala-Val (Ben-IKVAV) Synthesis

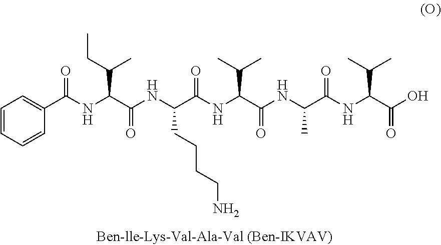 [SMILES: CCC(C)[C@H](NC(=O)c1ccccc1)C(=O)N[C@@H](CCCCN)C(=O)N[C@H](C(=O)N[C@@H](C)C(=O)N[C@H](C(=O)O)C(C)C)C(C)C]

A peptide derivative was prepared by using solid phase peptide synthesis (SPPS) of 2-chlorotrityl chloride resin (100 to 200 mesh and 0.3 to 0.8 mmol/g), the corresponding Fmoc-valine-OH, Fmoc-alanine-OH, Fmoc-valine-OH, Fmoc-lysine(Boc)-OH, Fmoc-isoleucine-OH and benzoic acid. First, the resin (0.65 g, 0.5 mmol) was swelled in anhydrous DCM for 30 minutes. Then, the C-terminal of the resin in anhydrous DMF and DIEA (0.28 mL, 1.67 mmol) was loaded with Fmoc-valine-OH (0.34 g, 1 mmol) for 1 hour. During the deprotection of Fmoc groups, 20% of piperidine in a DMF solution was used for 20 minutes, and then repeated twice (2 minutes for each time). Subsequently, Fmoc-alanine-OH (0.3 g, 1 mmol) was coupled to free amino for 40 minutes by using HBTU (0.38 g, 1 mmol) and DIEA (0.42 mL, 2.5 mmol) as coupling agents. After that, during the deprotection of Fmoc group, 20% of piperidine in a DMF solution was used for 20 minutes, and then repeated twice (2 minutes for each time). After that, Fmoc-valine-OH (0.34 g, 1 mmol) was coupled to free amino for 40 minutes by using HBTU (0.38 g, 1 mmol) and DIEA (0.42 mL, 2.5 mmol) as coupling agents. During the deprotection of Fmoc group, 20% of piperidine in a DMF solution was used for 20 minutes, and then repeated twice (2 minutes for each time). Subsequently, as described in the above steps, Fmoc-lysine(Boc)-OH (0.47 g, 1 mmol) and Fmoc-isoleucine-OH (0.35 g, 1 mmol) were coupled to free amino for 40 minutes by using HBTU (0.38 g, 1 mmol) and DIEA (0.42 mL, 2.5 mmol) as coupling agents. After that, during the deprotection of Fmoc groups, 20% of piperidine in a DMF solution was used for 20 minutes, and then repeated twice (2 minutes for each time). Finally, benzoic acid (0.19 g, 1.5 mmol) was coupled to free amino by using HBTU (0.55 g, 1.5 mmol) and DIEA (0.63 mL, 3.75 mmol) as coupling agents. The reaction mixture was stirred overnight and the excess reagent was removed by DMF, DCM, MeOH and hexane. The peptide derivative was cut off for 3 hours by using a 90% solution of trifluoroacetic acid in deionized water. The obtained solution was dried with air and was precipitated by adding ether to obtain the target product. The solid was vacuum dried to remove the remaining solution (white solid: 0.37 g). ¹H NMR (300 MHz, DMSO-d6): δ=0.8-2.15 (m, 32H), 2.7-2.85 (m, 2H), 4.15-4.50 (m, 5H), 7.45-8.0 (m, 10H), 8.08 (d, J=7.2 Hz, 1H), 8.22 (d, J=8.4 Hz, 1H), 8.37 (d, J=8.1 Hz, 1H); ¹³C NMR (75 MHz, DMSO-d6): δ=11.7, 16.4, 18.8, 19.1, 20.0, 20.1, 23.2, 25.8, 27.6, 31.0, 31.7, 32.2, 36.8, 48.9, 53.3, 58.0, 58.3, 58.9, 128.5, 129.2, 132.3, 135.2, 167.5, 171.3, 172.2, 173.2, 173.9; MS (EST⁺): calculated 660.4; measured 661.5 (M-H)⁺.

### Example 1-P

### Phenylacetic Acid-Ile-Lys-Val-Ala-Val (Benzyl-IKVAV) Synthesis

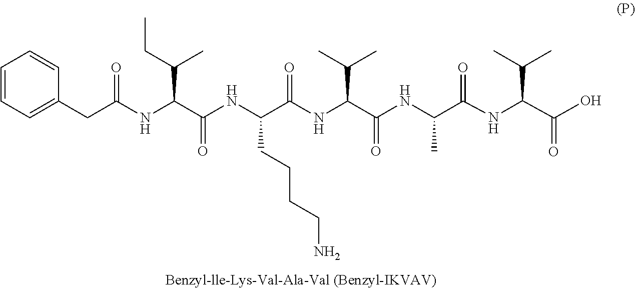 [SMILES: CCC(C)[C@H](NC(=O)Cc1ccccc1)C(=O)N[C@@H](CCCCN)C(=O)N[C@H](C(=O)N[C@@H](C)C(=O)N[C@H](C(=O)O)C(C)C)C(C)C]

A peptide derivative was prepared by using solid phase peptide synthesis (SPPS) of 2-chlorotrityl chloride resin (100 to 200 mesh and 0.3 to 0.8 mmol/g), the corresponding Fmoc-valine-OH, Fmoc-alanine-OH, Fmoc-valine-OH, Fmoc-lysine(Boc)-OH, Fmoc-isoleucine-OH andphenylacetic acid. First, the resin (1.21 g, 1 mmol) was swelled in anhydrous DCM for 30 minutes. Then, the C-terminal of the resin in anhydrous DMF and DIEA (0.83 mL, 5 mmol) was loaded with Fmoc-valine-OH (0.678 g, 2 mmol) for 1 hour. During the deprotection of Fmoc groups, 20% of piperidine in a DMF solution was used for 20 minutes, and then repeated twice (2 minutes for each time). Subsequently, Fmoc-alanine-OH (0.623 g, 2 mmol) was coupled to free amino for 40 minutes by using HBTU (0.758 g, 2 mmol) and DIEA (0.83 mL, 5 mmol) as coupling agents. After that, during the deprotection of Fmoc groups, 20% of piperidine in a DMF solution was used for 20 minutes, and then repeated twice (2 minutes for each time). After that, Fmoc-valine-OH (0.678 g, 2 mmol) was coupled to free amino for 40 minutes by using HBTU (0.758 g, 2 mmol) and DIEA (0.83 mL, 5 mmol) as coupling agents. During the deprotection of Fmoc groups, 20% of piperidine in a DMF solution was used for 20 minutes, and then repeated twice (2 minutes for each time). Subsequently, as described in the above steps, Fmoc-lysine(Boc)-OH (0.937 g, 2 mmol) and Fmoc-isoleucine-OH (0.707 g, 2 mmol) were coupled to free amino for 40 minutes by using HBTU (0.758 g, 2 mmol) and DIEA (0.83 mL, 5 mmol) as coupling agents. After that, during the deprotection of Fmoc groups, 20% of piperidine in a DMF solution was used for 20 minutes, and then repeated twice (2 minutes for each time). Finally, phenylacetic acid (0.45 g, 3 mmol) was coupled to free amino by using HBTU (1.1 g, 3 mmol) and DIEA (1.25 mL, 7.5 mmol) as coupling agents. The reaction mixture was stirred overnight and the excess reagent was removed by DMF, DCM, MeOH and hexane. The peptide derivative was cut off for 3 hours by using a 90% solution of trifluoroacetic acid in deionized water. The obtained solution was dried with air and was precipitated by adding ether to obtain the target product. The solid was vacuum dried to remove the remaining solution (white solid: 0.46 g). ¹H NMR (300 MHz, DMSO-d6): δ=0.8-2.2 (m, 32H), 2.766 (m, 2H), 2.85-3.15 (m, 2H), 4.10-4.50 (m, 5H), 7.15-7.40 (m, 5H), 7.6-8.25 (m, 7H); ¹³C NMR (75 MHz, DMSO-d6): δ=11.9, 16.3, 18.8, 19.1, 20.0, 20.1, 22.6, 23.2, 25.3, 27.5, 30.7, 30.9, 31.7, 32.1, 37.6, 43.0, 44.7, 48.9, 53.4, 57.9, 58.0, 58.2, 127.2, 129.1, 129.9, 137.6, 171.2, 171.3, 172.1, 172.3, 173.0, 173.2, 173.8, 171.7; MS (EST⁺): calculated 660.4; measured 661.5 (M-H)⁺.

### Example 1-Q

### 3-Phenylpropionic Acid-Ile-Lys-Val-Ala-Val (PropylBen-IKVAV) Synthesis

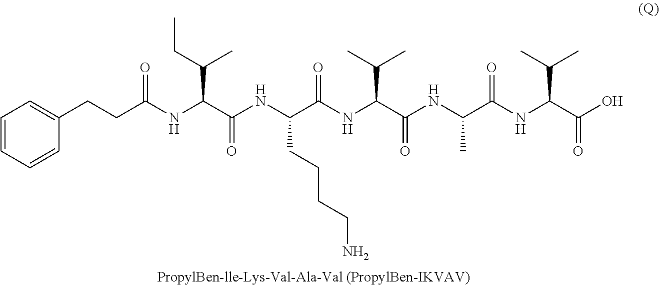 [SMILES: CCC(C)[C@H](NC(=O)CCc1ccccc1)C(=O)N[C@@H](CCCCN)C(=O)N[C@H](C(=O)N[C@@H](C)C(=O)N[C@H](C(=O)O)C(C)C)C(C)C]

A peptide derivative was prepared by using solid phase peptide synthesis (SPPS) of 2-chlorotrityl chloride resin (100 to 200 mesh and 0.3 to 0.8 mmol/g), the corresponding Fmoc-valine-OH, Fmoc-alanine-OH, Fmoc-valine-OH, Fmoc-lysine(Boc)-OH, Fmoc-isoleucine-OH and 3-phenylpropionic acid. First, the resin (1.21 g, 1 mmol) was swelled in anhydrous DCM for 30 minutes. Then, the C-terminal of the resin in anhydrous DMF and DIEA (0.83 mL, 5 mmol) was loaded with Fmoc-valine-OH (0.678 g, 2 mmol) for 1 hour. During the deprotection of Fmoc groups, 20% of piperidine in a DMF solution was used for 20 minutes, and then repeated twice (2 minutes for each time). Subsequently, Fmoc-alanine-OH (0.623 g, 2 mmol) was coupled to free amino for 40 minutes by using HBTU (0.758 g, 2 mmol) and DIEA (0.83 mL, 5 mmol) as coupling agents. After that, during the deprotection of Fmoc groups, 20% of piperidine in a DMF solution was used for 20 minutes, and then repeated twice (2 minutes for each time). After that, Fmoc-valine-OH (0.678 g, 2 mmol) was coupled to free amino for 40 minutes by using HBTU (0.758 g, 2 mmol) and DIEA (0.83 mL, 5 mmol) as coupling agents. During the deprotection of Fmoc groups, 20% of piperidine in a DMF solution was used for 20 minutes, and then repeated twice (2 minutes for each time). Subsequently, as described in the above steps, Fmoc-lysine(Boc)-OH (0.937 g, 2 mmol) and Fmoc-isoleucine-OH (0.707 g, 2 mmol) were coupled to free amino for 40 minutes by using HBTU (0.758 g, 2 mmol) and DIEA (0.83 mL, 5 mmol) as coupling agents. After that, during the deprotection of Fmoc groups, 20% of piperidine in a DMF solution was used for 20 minutes, and then repeated twice (2 minutes for each time). Finally, 3-phenylpropionic acid (0.45 g, 3 mmol) was coupled to free amino by using HBTU (1.1 g, 3 mmol) and DIEA (1.25 mL, 7.5 mmol) as coupling agents. The reaction mixture was stirred overnight and the excess reagent was removed by DMF, DCM, MeOH and hexane. The peptide derivative was cut off for 3 hours by using a 90% solution of trifluoroacetic acid in deionized water. The obtained solution was dried with air and was precipitated by adding ether to obtain the target product. The solid was vacuum dried to remove the remaining solution (white solid: 0.67 g). ¹H NMR (300 MHz, DMSO-d6): δ=1.15-2.2 (m, 32H), 2.961 (s, 2H), 3.15-3.3 (m, 4H), 4.55-4.90 (m, 5H), 7.60-7.75 (m, 5H), 8.1-8.3 (m, 4H), 8.33 (d, J=8.7 Hz, 1H), 8.4 (d, J=8.7 Hz, 1H), 8.5 (d, J=7.5 Hz, 1H), 8.55 (d, J=8.4 Hz, 1H); ¹³C NMR (75 MHz, DMSO-d6): δ=11.8, 16.3, 18.8, 19.1, 20.0, 20.1, 23.2, 25.3, 27.5, 30.9, 32.1, 32.2, 37.3, 37.6, 48.9, 53.3, 57.8, 58.0, 58.2, 126.8, 129.2, 142.2, 171.3, 172.2, 172.2, 172.4, 173.2, 173.8, 207.5; MS (EST⁺): calculated 660.4; measured 661.5 (M-H)⁺.

### Example 1-R

### PFB-Phe-Ile-Lys-Val-Ala-Val (PFB-FIKVAV) Synthesis

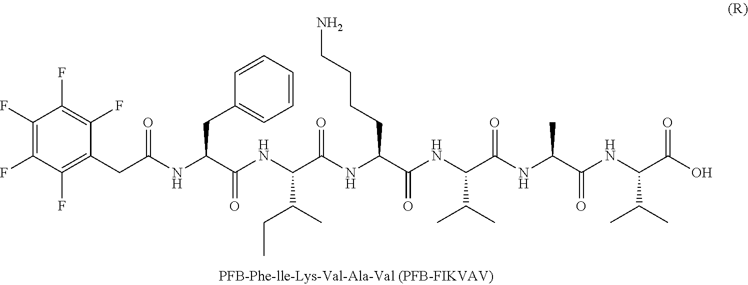 [SMILES: CCC(C)[C@H](NC(=O)[C@H](Cc1ccccc1)NC(=O)Cc1c(F)c(F)c(F)c(F)c1F)C(=O)N[C@@H](CCCCN)C(=O)N[C@H](C(=O)N[C@@H](C)C(=O)N[C@H](C(=O)O)C(C)C)C(C)C]

A peptide derivative was prepared by using solid phase peptide synthesis (SPPS) of 2-chlorotrityl chloride resin (100 to 200 mesh and 0.3 to 0.8 mmol/g), which was treated with Fmoc-valine-OH, Fmoc-alanine-OH, Fmoc-valine-OH, Fmoc-lysine(Boc)-OH, Fmoc-isoleucine-OH, Fmoc-L-phenylalanine and pentafluorophenyl acetic acid. First, 1.2 g of resin was swelled in anhydrous DCM for 30 minutes. Then, the resin in anhydrous DMF and DIEA (0.83 mL, 5 mmol) was loaded with Fmoc-valine-OH (0.678 g, 2 mmol) for 1 hour. During the deprotection of Fmoc groups, 20% of piperidine in a DMF solution was used for 20 minutes, and then repeated twice (2 minutes for each time). Subsequently, Fmoc-alanine-OH (0.623 g, 2 mmol) was coupled to free amino for 40 minutes by using HBTU (0.758 g, 2 mmol) and DIEA (0.83 mL, 5 mmol) as coupling agents. After that, during the deprotection of Fmoc groups, 20% of piperidine in a DMF solution was used for 20 minutes, and then repeated twice (2 minutes for each time). Fmoc-valine-OH (0.678 g, 2 mmol) was coupled to free amino for 40 minutes by using HBTU (0.758 g, 2 mmol) and DIEA (0.83 mL, 5 mmol) as coupling agents. During the deprotection of Fmoc group, 20% of piperidine in a DMF solution was used for 20 minutes, and then repeated twice (2 minutes for each time). After that, Fmoc-lysine(Boc)-OH (0.937 g, 2 mmol) and Fmoc-isoleucine-OH (0.707 g, 2 mmol) were used to repeat the above steps for 40 minutes by using HBTU (0.758 g, 2 mmol) and DIEA (0.83 mL, 5 mmol) as coupling agents. During the deprotection of Fmoc groups, 20% of piperidine in a DMF solution was used for 20 minutes, and then repeated twice (2 minutes for each time). Subsequently, Fmoc-L-phenylalanine (0.78 g, 2 mmol) was coupled to free amino for 40 minutes by using HBTU (0.76 g, 2 mmol) and DIEA (0.83 mL, 5 mmol) as coupling agents. During the deprotection of Fmoc groups, 20% of piperidine in a DMF solution was used for 20 minutes, and then repeated twice (2 minutes for each time). Finally, pentafluorophenyl acetic acid (0.79 g, 3.5 mmol) was coupled to free amino by using HBTU (1.3 g, 3.5 mmol) and DIEA (1.45 mL, 8.75 mmol) as coupling agents. The reaction mixture was stirred overnight and the excess reagent was removed by DMF, DCM, MeOH and hexane. The peptide derivative was cut off for 3 hours by using a 90% solution of trifluoroacetic acid in deionized water. The obtained solution was dried with air and was precipitated by adding ether to obtain the target product. The solid was vacuum dried to remove the remaining solution (white solid: 0.72 g). ¹H NMR (300 MHz, DMSO-d6): δ=0.8-2.15 (m, 32H), 2.70-3.10 (m, 4H), 3.62 (s, 2H), 4.10-4.70 (m, 6H), 7.26 (d, J=5.1 Hz, 5H), 7.65-7.85 (m, 4H), 7.9 (d, J=8.7 Hz, 1H), 7.99 (d, J=8.4 Hz, 1H), 8.07 (d, J=7.5 Hz, 1H), 8.13 (d, J=7.8 Hz, 1H), 8.53 (d, J=8.7 Hz, 1H); ¹³C NMR (75 MHz, DMSO-d6): δ=11.0, 15.3, 17.8, 18.2, 19.0, 19.1, 22.2, 24.2, 26.6, 28.6, 30.0, 30.7, 31.3, 36.8, 37.3, 38.7, 39.0, 47.9, 52.3, 54.0, 56.9, 57.0, 57.2, 110.2, 116.1, 118.5, 126.2, 127.9, 129.2, 135.7, 137.7, 143.9, 145.8, 166.6, 170.3, 170.7, 171.2, 172.3, 172.8; MS (EST⁺): calculated 883.4; measured 884.2.

### Example 2

### Preparation of Hydrogels (A) to (R) from the Peptide Molecular Materials (A) to (R) Prepared in Example 1

Hydrogel (A): 3.3 mg of PFB-F was dissolved in 270 μL of water and was homogenized by ultrasonication. After 2 μL of 1 M sodium hydroxide was added and dissolved, 16 μL of 0.1 M hydrochloric acid was added and the solution was allowed to stand overnight.

Hydrogel (B): 3.1 mg of PFB-FG was dissolved in 280 μL of water and was homogenized by ultrasonication. After 2 μL of 0.5 M sodium hydroxide was added and dissolved, 2 μL of 0.5M hydrochloric acid was added (white solid was produced). Further, 2 μL of 0.5 M sodium hydroxide and 2 μL of 0.1 M sodium hydroxide were added and followed by the addition of 10 μL of 0.1 M hydrochloric acid.

Hydrogel (C): 2.1 mg of PFB-YF was dissolved in 180 μL of water and was homogenized by ultrasonication. After 2 μL of 0.5 M sodium hydroxide was added and dissolved, 6 μL of 0.1M hydrochloric acid and 2 μL of 0.05 M hydrochloric acid were added.

Hydrogel (D): 1.9 mg of PFB-FF was dissolved in 180 μL of water and was homogenized by ultrasonication. After 4 μL of 0.5 M sodium hydroxide was added and dissolved, 6 μL of 0.1 M hydrochloric acid was added.

Hydrogel (E): 2.2 mg of PFB-FA was dissolved in 185 μL of water and was ultrasonicated. After 4 μL of 0.5 M sodium hydroxide was added and dissolved, 12 μL of 0.1 M hydrochloric acid, 4 μL of 0.1 M sodium hydroxide and 2 μL of 0.1 M hydrochloric acid were added sequentially.

Hydrogel (F): 1.9 mg of PFB-FV was dissolved in 185 μL of water and was ultrasonicated. After 4 μL of 0.5 M sodium hydroxide was added and dissolved and 4 μL of 0.1 M hydrochloric acid was added, the addition of 2 μL of 0.1 M sodium hydroxide and 2 μL of 0.1 M hydrochloric acid was repeated 3 times and then the solution was allowed to stand overnight.

Hydrogel (G): 2.0 mg of PFB-VF was dissolved in 180 μL water and was ultrasonicated. 6 μL of 1 M sodium hydroxide, 2 μL of 0.5 M sodium hydroxide, 4 μL of 1 M hydrochloric acid and 4 μL of 0.1 M sodium hydroxide were added sequentially.

Hydrogel (H): 2.2 mg of PFB-IF was dissolved in 180 μL of water and was ultrasonicated. 4 μL of 1 M sodium hydroxide, 6 μL of 0.5 M sodium hydroxide, 4 μL of 1 M hydrochloric acid, 2 μL of 0.1 M hydrochloric acid and 4 μL of 0.5 M hydrochloric acid were added sequentially.

Hydrogel (I): 2.0 mg of PFB-LF was dissolved in 180 μL of water and was ultrasonicated. 4 μL of 1 M sodium hydroxide and 6 μL of 0.5 M hydrochloric acid were added sequentially.

Hydrogel (J): 2 mg of PFB-_{D}-L-_{D}-F was dissolved in 180 μL of water and was ultrasonicated. After 10 μL of 1 M sodium hydroxide was added, 8 μL of 1 M hydrochloric acid were added and the solution was allowed to stand overnight.

Hydrogel (K): 4 mg of 4-MFB-FF was dissolved in 150 μL of water and was ultrasonicated. After 22 μL of 0.5 M sodium hydroxide was added, 2 μL of 0.5 M hydrochloric acid and 30 μL of 0.1 M hydrochloric acid were added and the solution was allowed to stand overnight.

Hydrogel (L): 2 mg of PFB-GFF was dissolved in 180 μL of water and was ultrasonicated. After 4 μL of 0.5 M sodium hydroxide was added, 2 μL of 0.5 M hydrochloric acid and 10 μL of 0.1 M hydrochloric acid were added and the solution was allowed to stand overnight.

Hydrogel (M): 2 mg of PFB-IKVAV was dissolved in 180 μL of water and was ultrasonicated. 10 μL of 0.5 M sodium hydroxide and 10 μL of 0.5 M hydrochloric acid were added.

Hydrogel (N): 2.1 mg of 4-MFB-IKVAV was dissolved in 180 μL of water and was ultrasonicated. 4 μL of 1 M sodium hydroxide and 12 μL of water were added.

Hydrogel (O): 2 mg of Ben-IKVAV was dissolved in 200 μL of water and was ultrasonicated.

Hydrogel (P): 2 mg of Benzyl-IKVAV was dissolved in 200 μL of water and was ultrasonicated.

Hydrogel (Q): 2.1 mg of PropylBen-IKVAV was dissolved in 200 μL of water and was ultrasonicated.

Hydrogel (R): 2.2 mg of PFB-FIKVAV was dissolved in 180 μL of water. 2 μL of 0.5 M sodium hydroxide was added and was ultrasonicated.

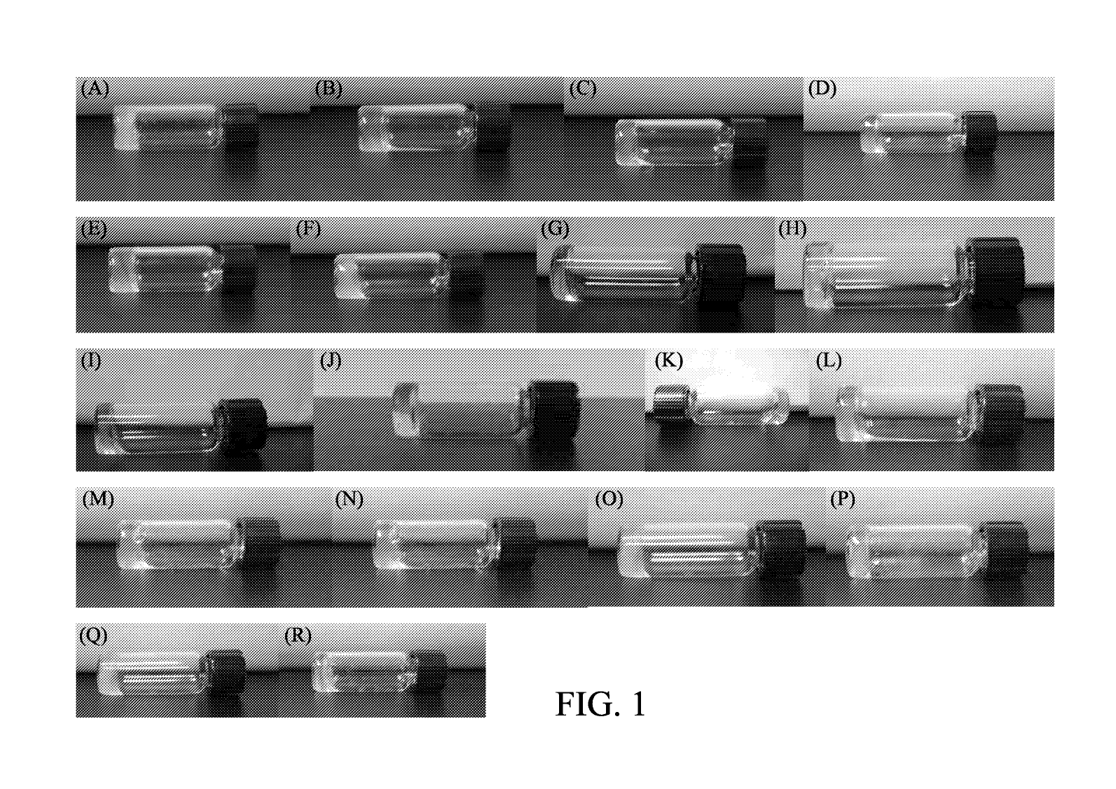

FIG. 1 shows the optical images of hydrogels (A) PFB-F (1 wt %, pH=5), (B) PFB-FG (1 wt %, pH=6-7), (C) PFB-YF (1 wt %, pH=7), (D) PFB-FF (1 wt %, pH=9-10), (E) PFB-FA (1 wt %, pH=6-7), (F) PFB-FV (1 wt %, pH=6-7), (G) PFB-VF (1 wt %, pH=⁷), (H) PFB-IF (pH=7), (I) PFB-LF (pH=7), (J) PFB-_{D}-L-_{D}-F (1 wt %, pH=7-8), (K) 4-MFB-FF (2 wt %, pH=7-8), (L) PFB-GFF (1 wt %, pH=5-6), (M) PFB-IKVAV (1 wt %, pH=7-8), (N) 4-MFB-IKVAV (1 wt %, pH=9), (O) Ben-IKVAV (1 wt %, pH=2-4), (P) Benzyl-IKVAV (1 wt %, pH=4), (Q) PropylBen-IKVAV, and (R) PFB-FIKVAV (1 wt %, pH=5-8).

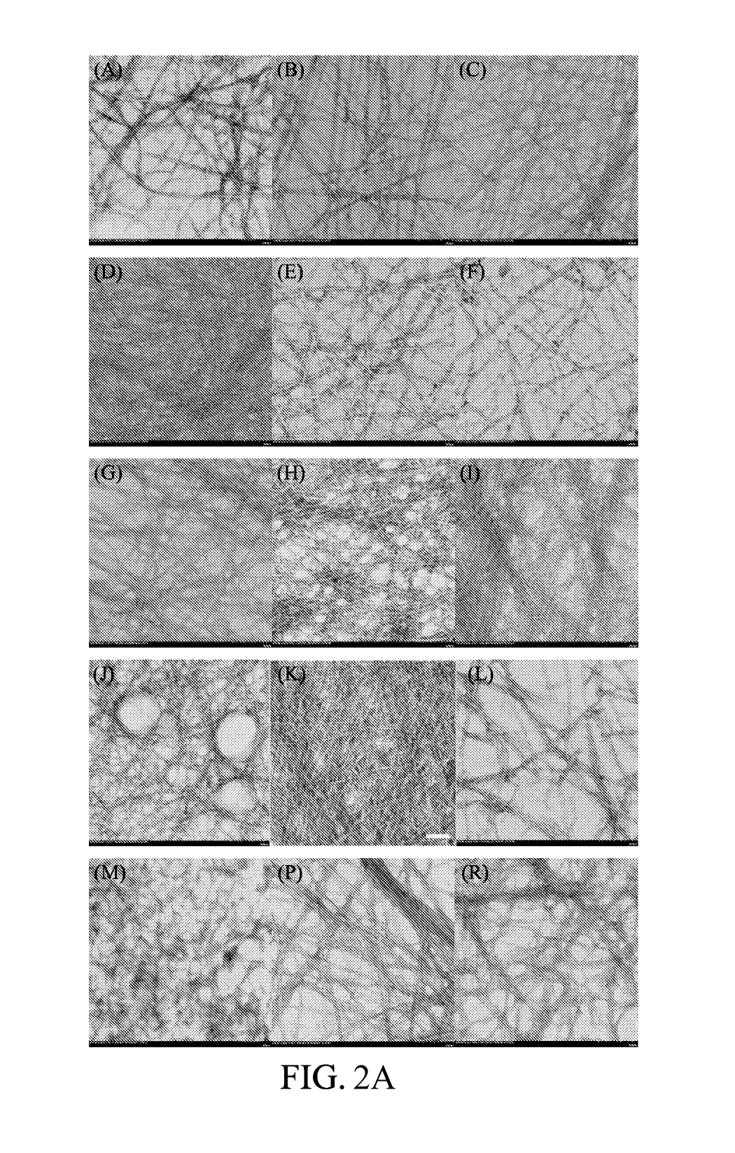

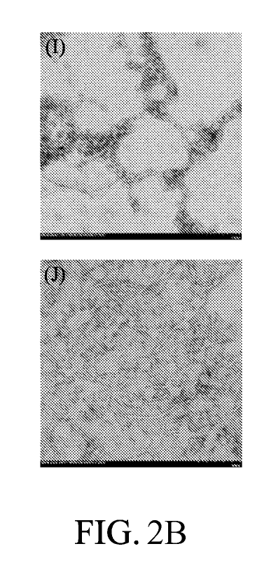

FIG. 2A shows the transmission electron microscopy images of hydrogels (A) PFB-F, (B) PFB-FG, (C) PFB-YF, (D) PFB-FF, (E) PFB-FA, (F) PFB-FV, (G) PFB-VF, (H) PFB-IF (pH=7), (I) PFB-LF (pH=7), (J) PFB-_{D}-L-_{D}-F (pH=7.2), (K) 4-MFB-FF, (L) PFB-GFF, (M) PFB-IKVAV, (P) Benzyl-IKVAV and (R) PFB-FIKVAV at 37° C. As shown in FIG. 2A, the hydrogel of the present invention forms a three-dimensional reticular structure through the self-assembled ability (e.g., hydrogen bond, π-π interaction, van der Waals force and solvation effect) between peptide molecules.

FIG. 2B shows the transmission electron microscopy images of hydrogels (I) PFB-LF and (L) PFB-_{D}-L-_{D}-F at 4° C. As shown in FIG. 2B, hydrogels (I) PFB-LF and (L) PFB-_{D}-L-_{D}-F of the present invention can be liquid. Therefore, by using the properties of hydrogels (I) and (L) that have different states at different temperatures, hydrogels (I) and (L) are in a colloidal state at 37° C., so as to encapsulate a substance and the hydrogels become a liquid state when the temperature is down to 4° C., so as to release the substance.

### Example 3

### Rheological Tests of Hydrogels

Rheological tests of hydrogels were performed by Anton Paar rheometer. 25 mm parallel plate was used in the experimentation. 400 μL of hydrogels (A) to (R) were placed on the parallel plate. Angular frequency sweep test: measurement range (frequency 0.1 to 100 rad/s, strain=0.8%) is 13 points per 10 rounds. Sweep model is “logarithm (log)” and the operation temperature is 25° C.

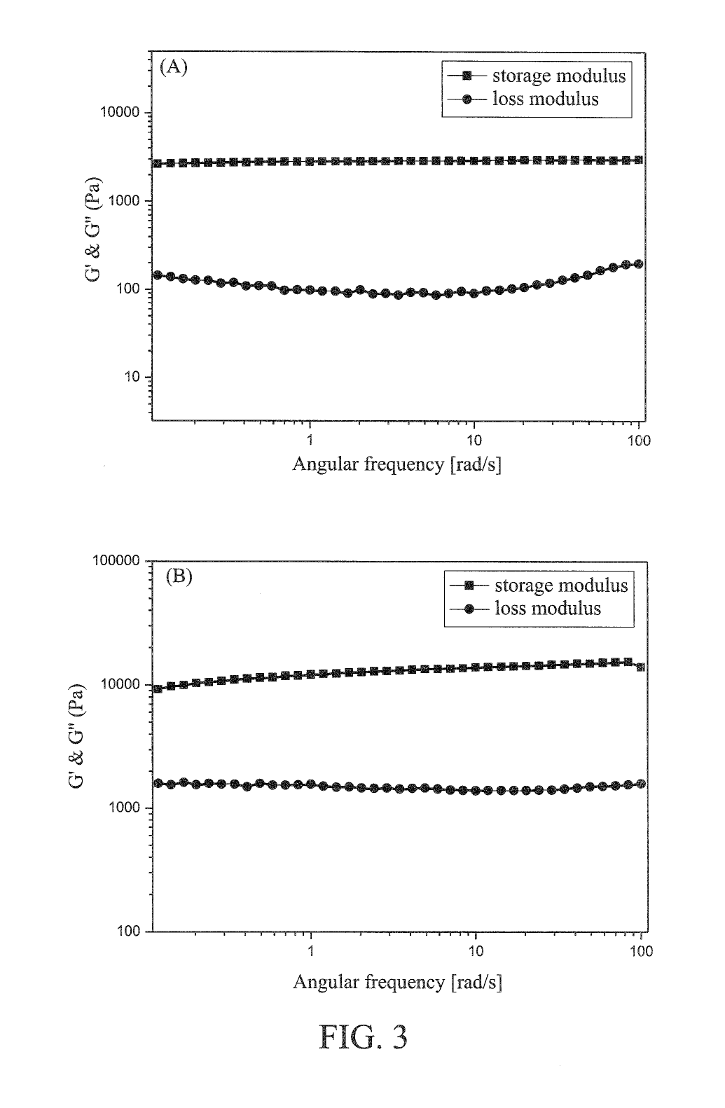

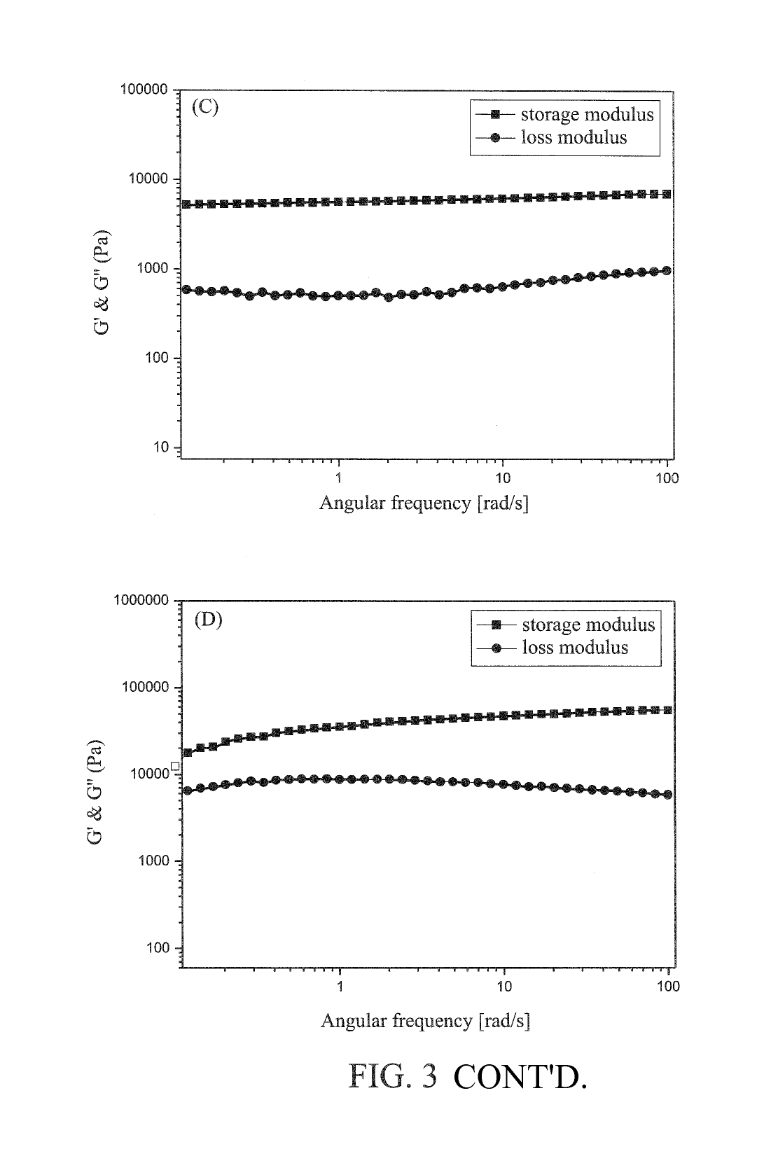

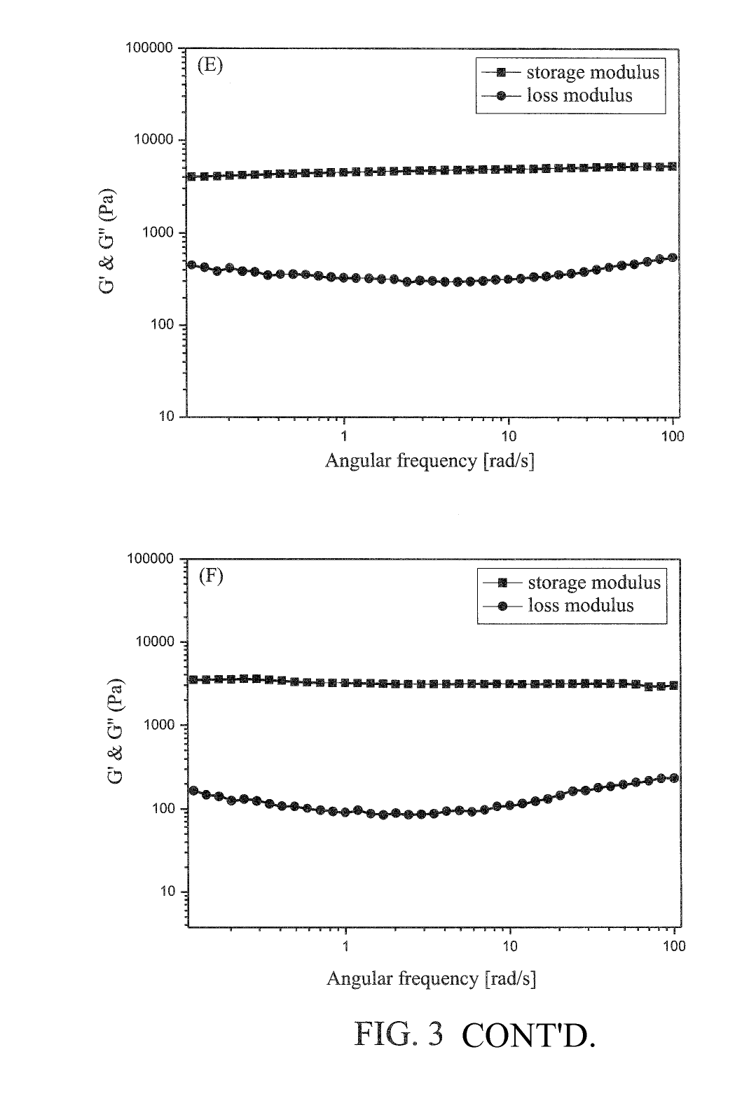

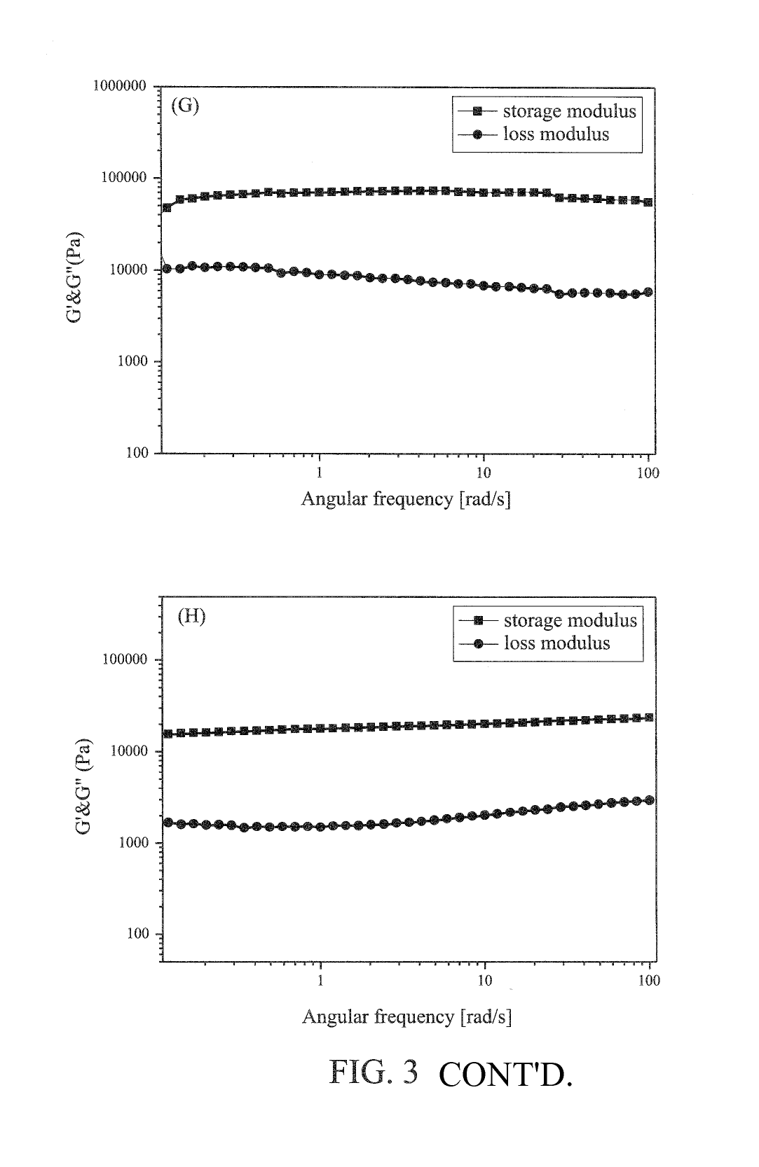

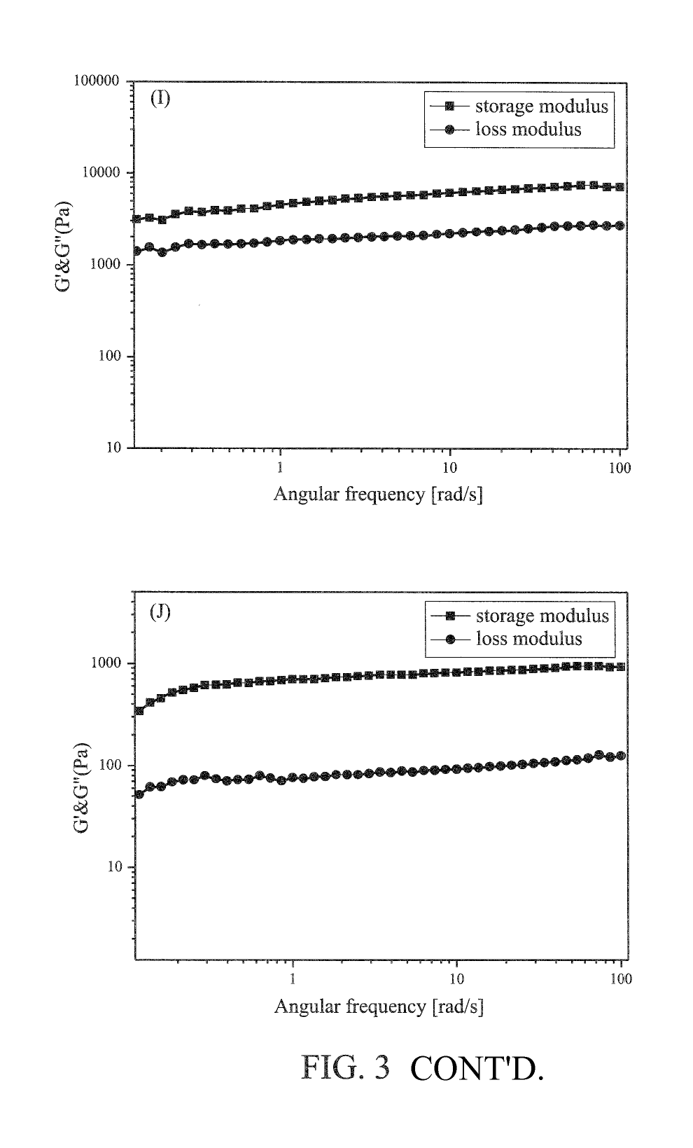

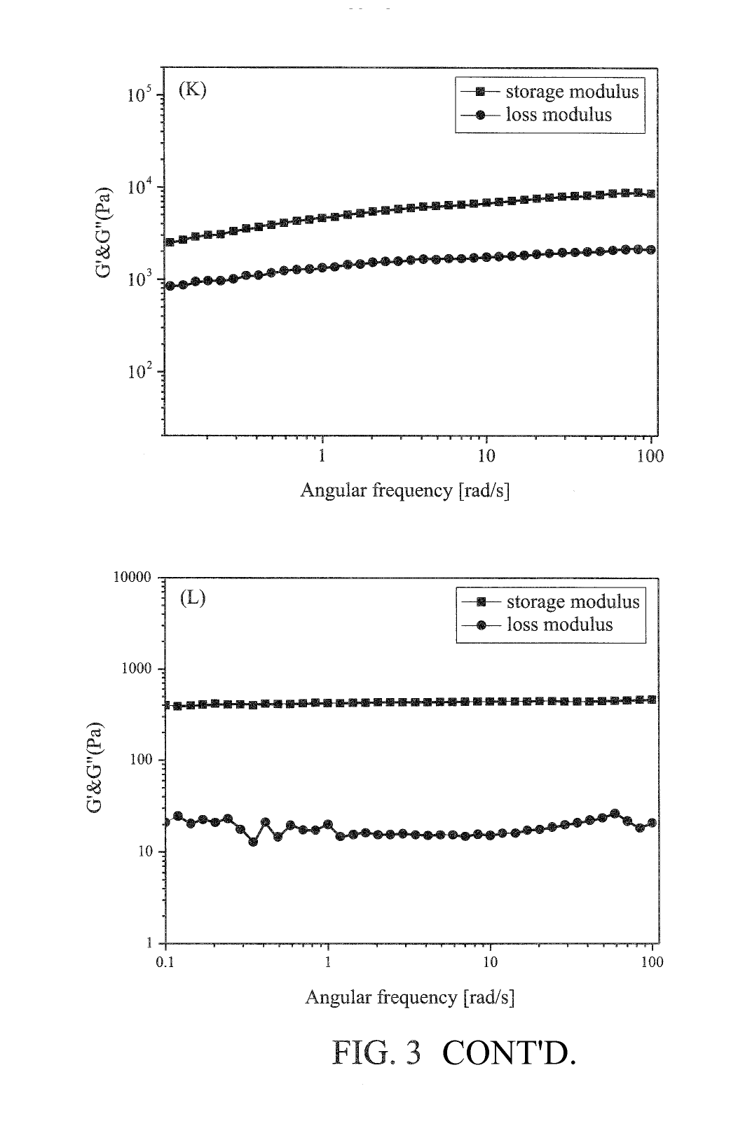

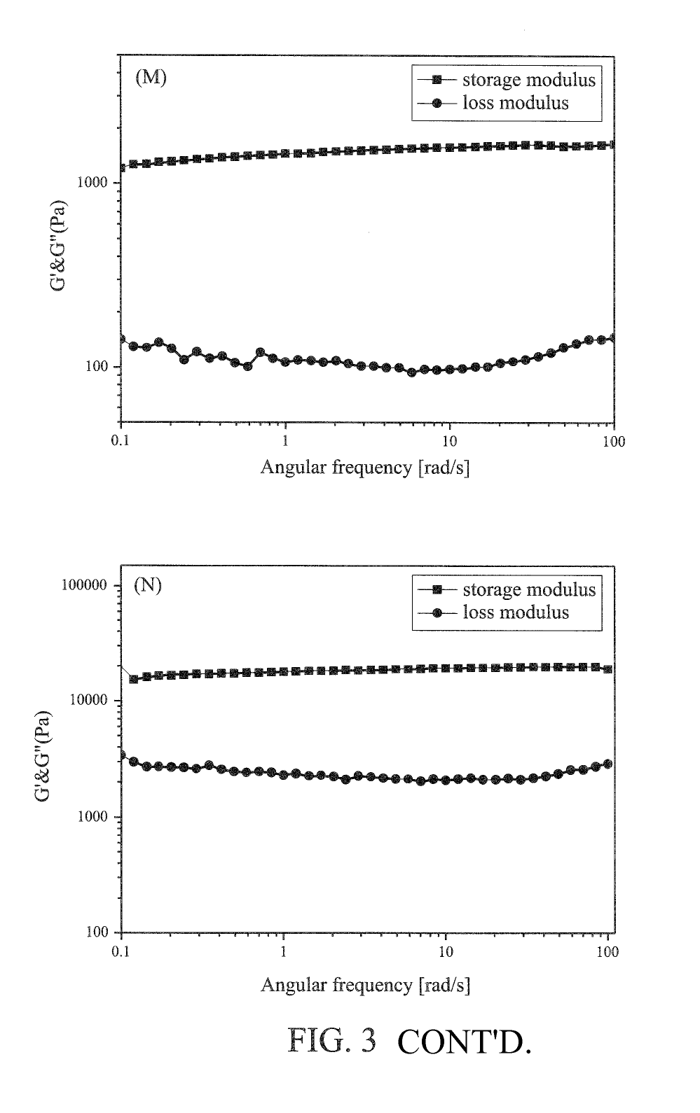

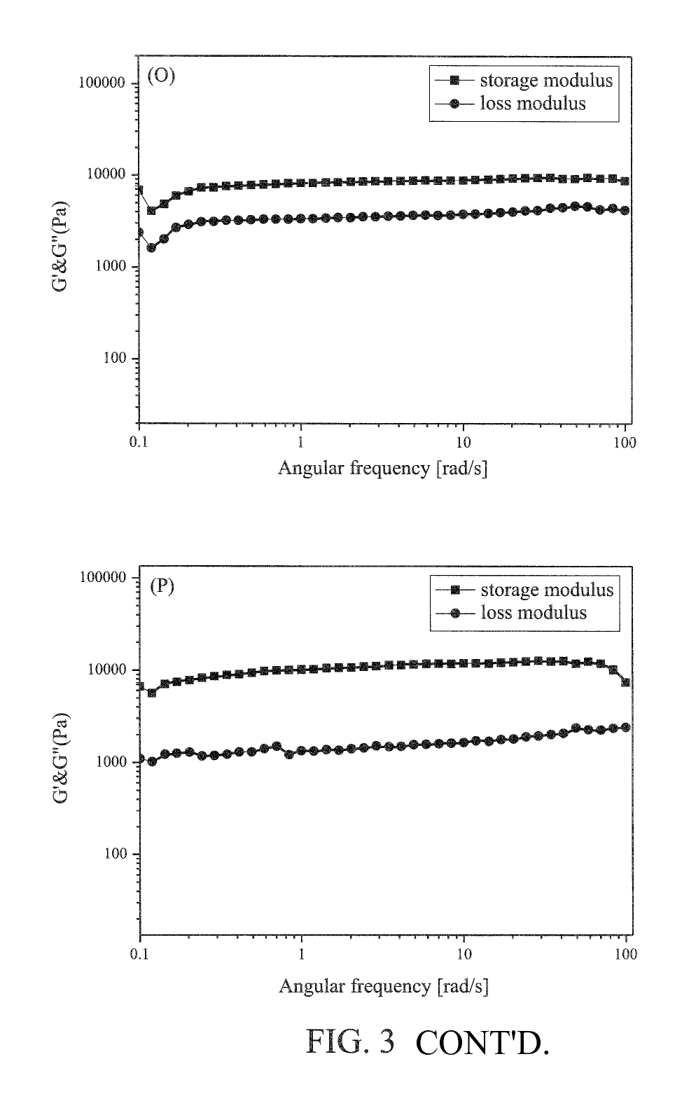

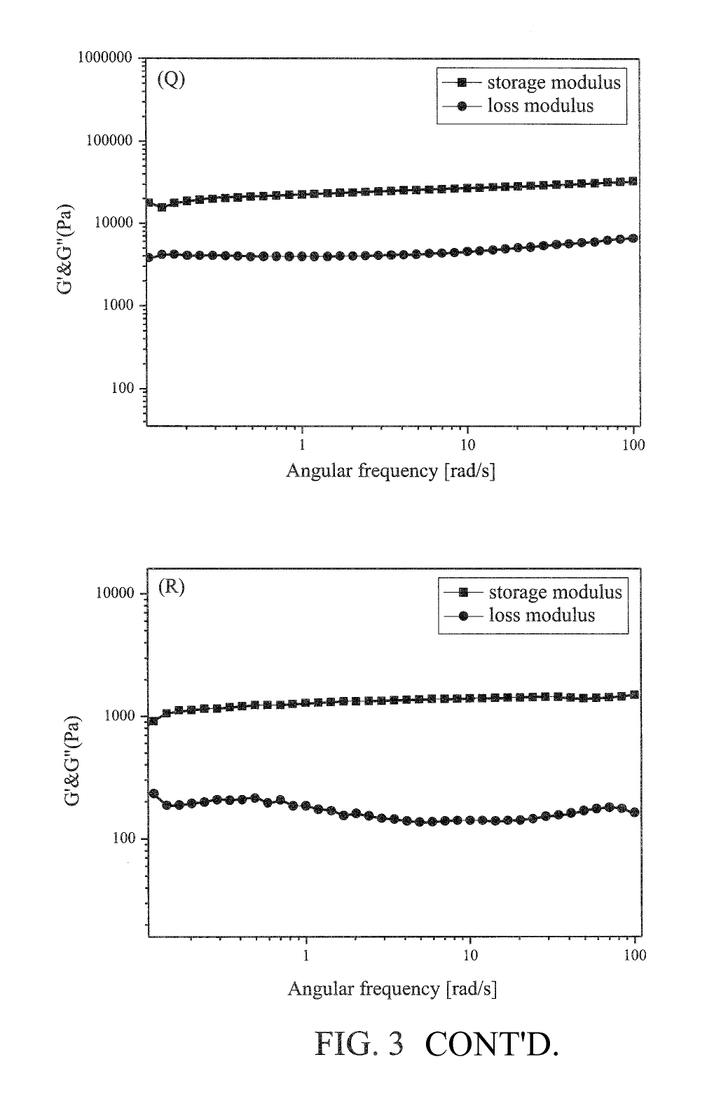

FIG. 3 shows the relationship between the storage modulus and the loss modulus of hydrogels (A) to (R). In FIG. 3, G′ represents storage modulus, and G″ represents loss modulus. The higher G′ and G″ are, the better stability the hydrogel is.

In the measurement of Angular frequency of from 0.1% to 100%, it could be seen that the storage modulus of hydrogel (A) was 2×10³; the storage modulus of hydrogel (B) was 10⁴; the storage modulus of hydrogel (C) was 5×10³; the storage modulus of hydrogel (D) was 2×10⁴; the storage modulus of hydrogel (E) was 4×10³; the storage modulus of hydrogel (F) was 3×10³; the storage modulus of hydrogel (G) was 6×10⁴; the storage modulus of hydrogel (H) was 1×10⁴; the storage modulus of hydrogel (I) was 2×10³; the storage modulus of hydrogel (J) was 7×10²; the storage modulus of hydrogel (k) was 6×10³; the storage modulus of hydrogel (L) was 4×10²; the storage modulus of hydrogel (M) was 1×10³; the storage modulus of hydrogel (N) was 1×10⁴; the storage modulus of hydrogel (O) was 6×10³; the storage modulus of hydrogel (P) was 6×10³; the storage modulus of hydrogel (Q) was 1×10⁴; and the storage modulus of hydrogel (R) was 1.0×10³. The above results show that the storage moduli were larger than the minimum energy modulus for supporting cells (100 Pa).

### Example 4

### The Phase Inversion Temperature (T_{gel-sol}) Tests of Hydrogels

Hydrogels (A) to (R) were obliquely placed in water bath while a beaker including water and a thermometer was also placed in the water bath. The hydrogels were heated (2° C./min) until such hydrogels began to flow (the gel phase was changed to the sol phase). The inversion temperatures when the hydrogels began to flow were recorded, and such temperatures were the phase inversion temperatures of hydrogels.

The T_{gel-sol} of such hydrogels are as follows. The T_{gel-sol} of hydrogel (A) was 56° C., the T_{gel-sol} of hydrogel (B) was 48° C., the T_{gel-sol} of hydrogel (C) was 43° C., the T_{gel-sol} hydrogel (D) was 45° C., the T_{gel-sol} of hydrogel (E) was 48° C., the T_{gel-sol} of hydrogel (F) was 46° C., the T hydrogel (G) was 72° C., the T_{gel-sol} of hydrogel (H) was 71° C., the T_{gel-sol} of hydrogel (I) was 55° C., the T_{gel-sol} of hydrogel (J) was 69° C., the T_{gel-sol} of hydrogel (K) was 46° C., the T_{gel-sol} of hydrogel (L) was 38° C., the T_{gel-sol} of hydrogel (M) was 42° C., the

T_{gel-sol} of hydrogel (N) was 66° C., the T_{gel-sol} of hydrogel (O) was 62° C., the T_{gel-sol} of hydrogel (P) was 65° C., the T_{gel-sol} of hydrogel (Q) was >90° C., and the T_{gel-sol} of hydrogel (R) was 40° C. As a whole, all T_{gel-sol} are more than 38° C. Thus, the results show that such hydrogels had excellent stability in the human body temperature.

### Example 5

### Cell Viability Assay

The bio-compatibility of the hydrogels prepared from the different peptide materials was measured by MTT cell viability assay.

MTT cell viability assay was performed by Sunrise absorbance microplate reader (DV990/BV4 GDV Programmable MPT reader). Various cells were seeded in a 24-well plate containing 0.5 mL medium (DMEM) with 10% fetal bovine serum (FBS) and 1% penicillin. In each well, the cell concentration was 50000 cells and cells were cultured for 24 hours. After seeding the cells, hydrogels with the different concentrations (10, 50, 100, 200, 500 mM) were added. The medium in each well was replaced with the flesh medium containing 0.5 mL of MTT reagent (4 mg mL⁻¹) after 24 and 48 hours. After an additional 4 hours, the medium containing MTT reagent was removed and DMSO (0.5 mL/well) was added to dissolve formazan crystals. The cells in the 24-well plate were transferred to a 96-well plate. The optical densities of such obtained solutions were measured at 595 nm by Sunrise absorbance microplate reader (DV990/BV4 GDV Programmable MPT reader).

The untreated cells were used as a comparative example. The cell viability was calculated based on the following equation:

Cell viability(%)=ODₛₐₘₚₗₑ/OD_{comparative example}

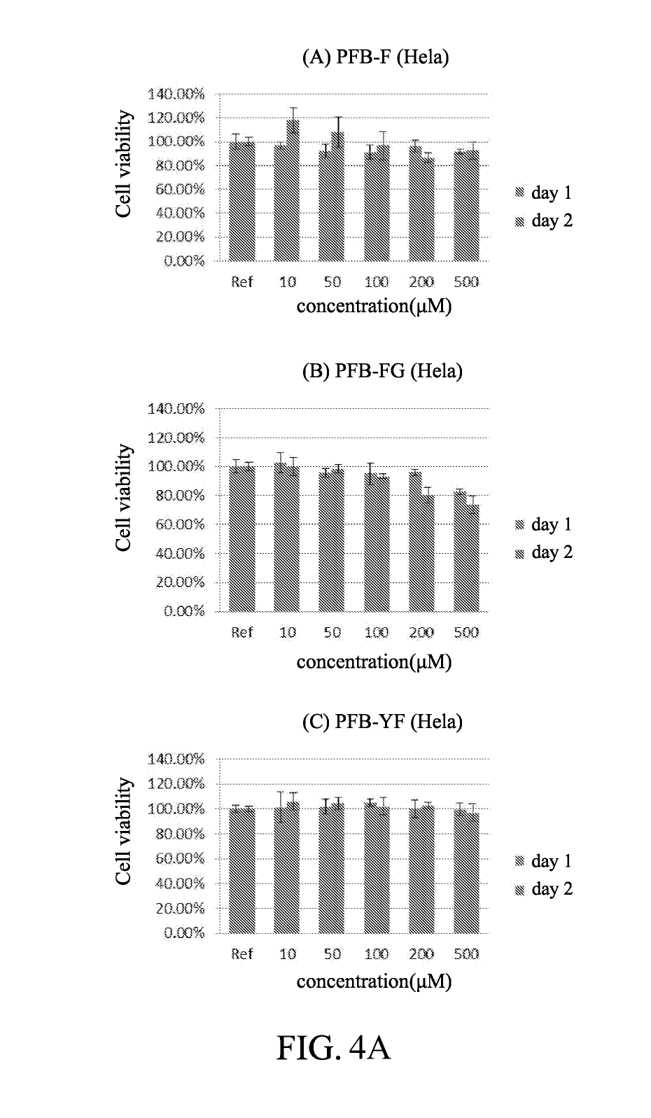

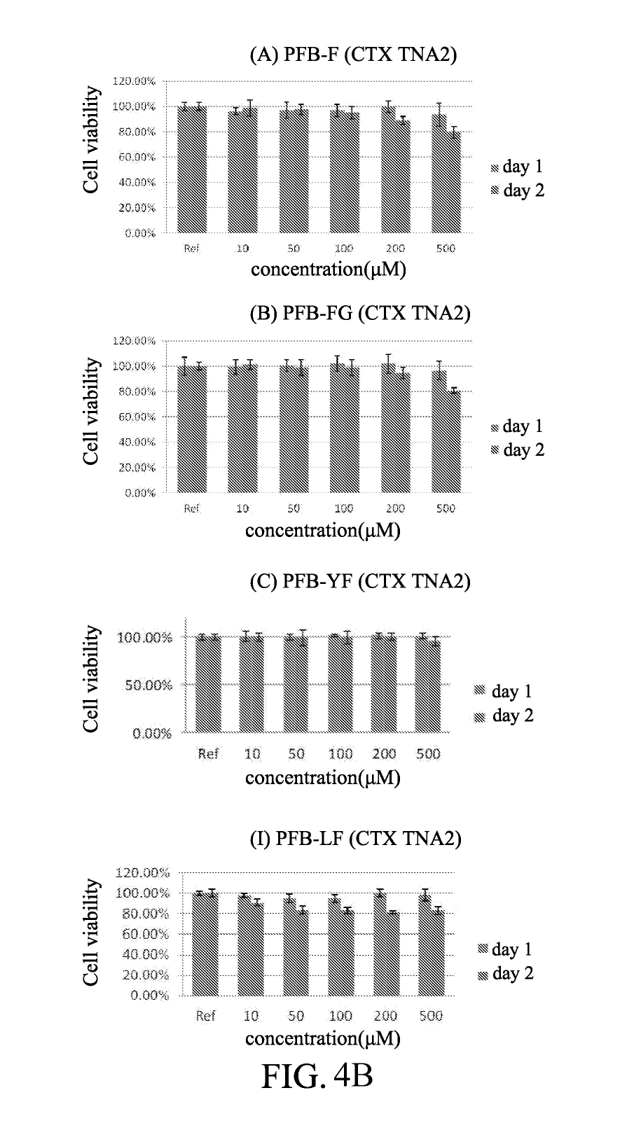

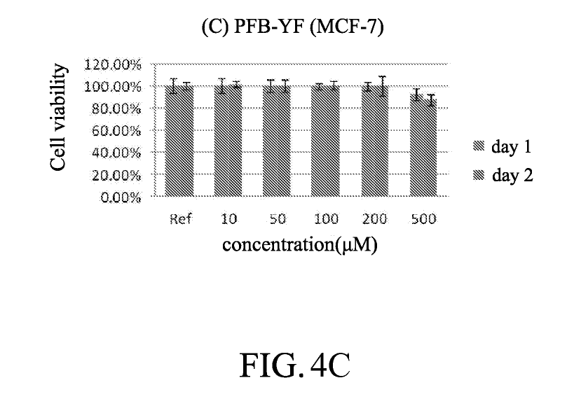

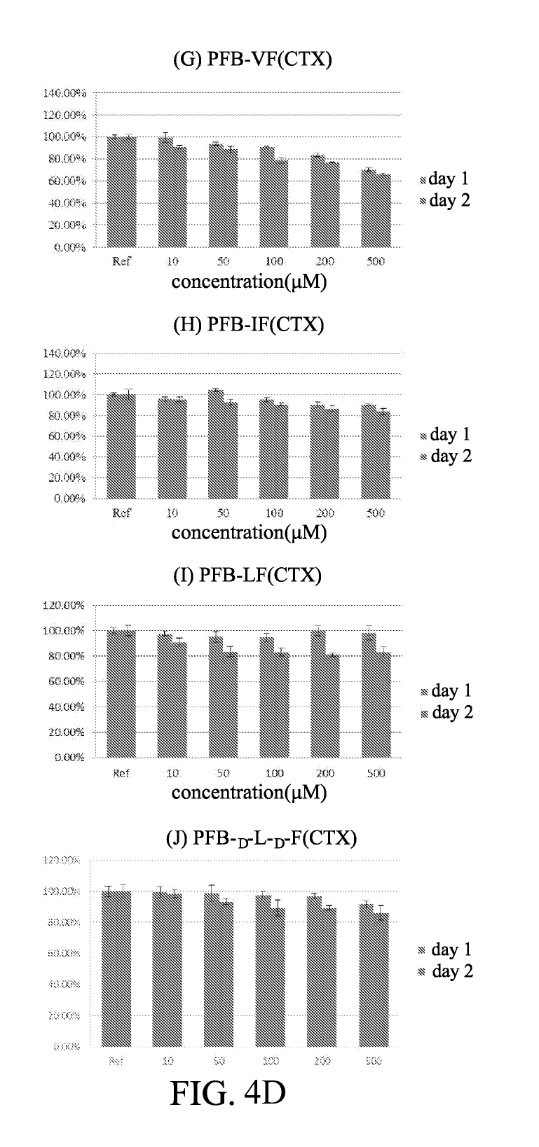

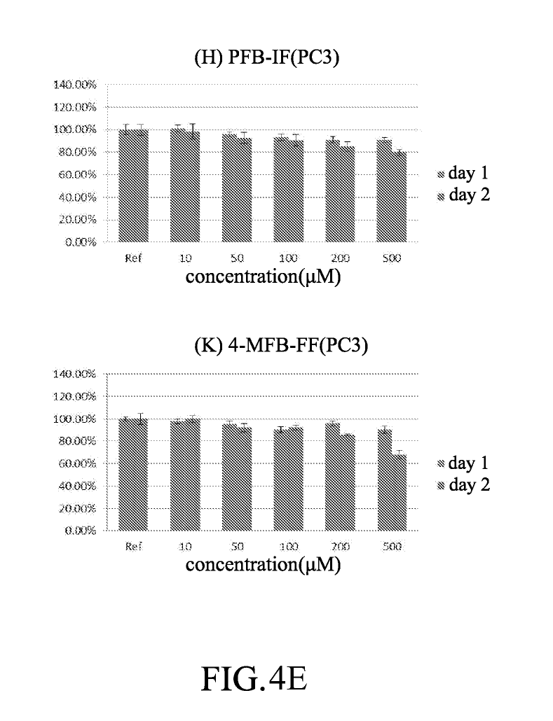

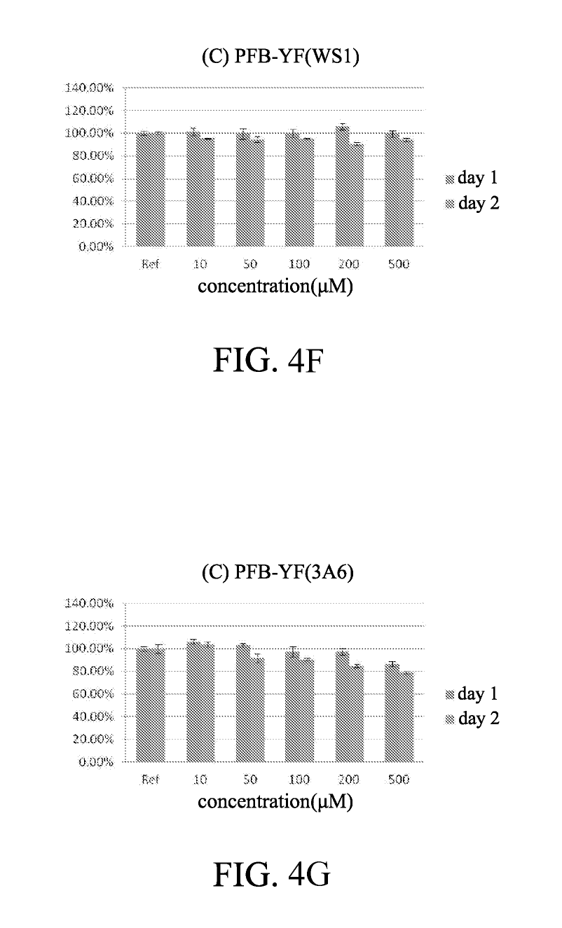

FIG. 4A shows the Hela cell viability assays of hydrogels (A), (B) and (C). FIG. 4B shows the CTX TNA2 cell viability assays of hydrogels (A), (B), (C) and (I). FIG. 4C shows the MCF-7 cell viability assay of hydrogel (C). FIG. 4D shows the CTX cell viability assay of hydrogel (G), (H), (I) and (J). FIG. 4E shows the PC3 cell viability assay of hydrogel (H) and (K). FIG. 4F shows the WS1 cell viability assay of hydrogel (C). FIG. 4G shows the 3A6 cell viability assay of hydrogel (C). From the results of the cell viability assays, it was found that the cell viability at 500 μM of hydrogel achieved 80%. Especially, the Hela, CTX TNA2 and MCF-7 cell viability results of hydrogel (C) were approximately 100%. That is, their IC₅₀ inhibition concentrations were more than 500 μM. The results show that such hydrogels were hydrogel materials having bio-compatibility (no cytotoxicity).

### Example 6

### Wound Healing Assay

Various cells were washed with PBS (phosphate buffer) twice and were suspended in a T-75 tissue culture flask. 0.25% trypsin containing 0.1% EDTA was added and then the cells were re-suspended in 5 mL of complete medium. 30000 cells (in 3 mL of medium) were placed in each vial on a 6-well plate to form a fusion monolayer. After the adhesion for 24 hours, the cell monolayer was scratched by a p200 pipet tip to create a wound. 2 mL of PBS was used twice to remove the floating cells, and was then replaced with 3 mL of complete medium. The image taken at 0 hour was used as a reference point. The medium was replaced with 3 mL of medium containing 1 wt % of hydrogel and the plate was incubated at 37° C. and 5% CO₂ for 24 hours. The images at different hours were taken at an appropriate region. Control: no compound added.

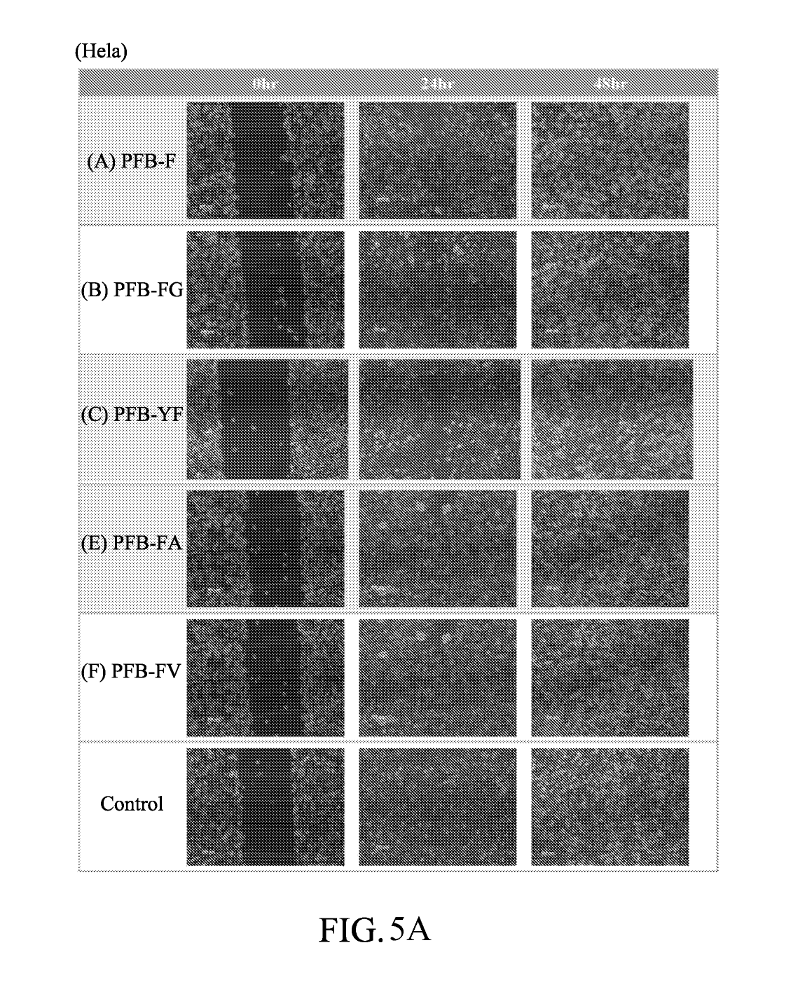

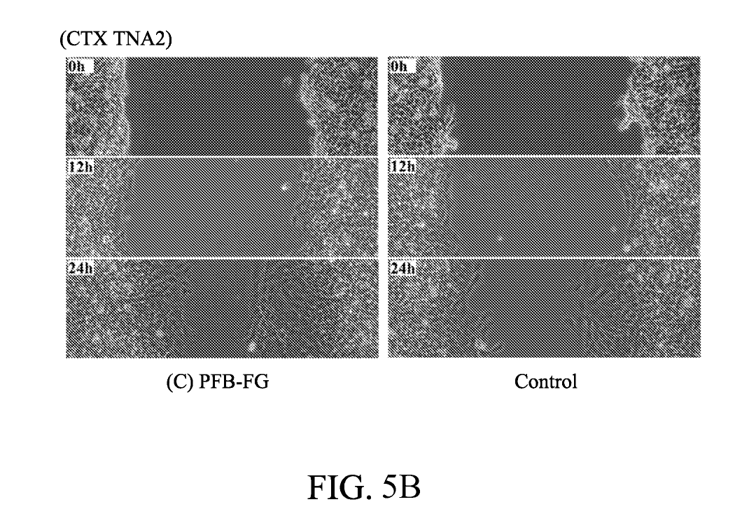

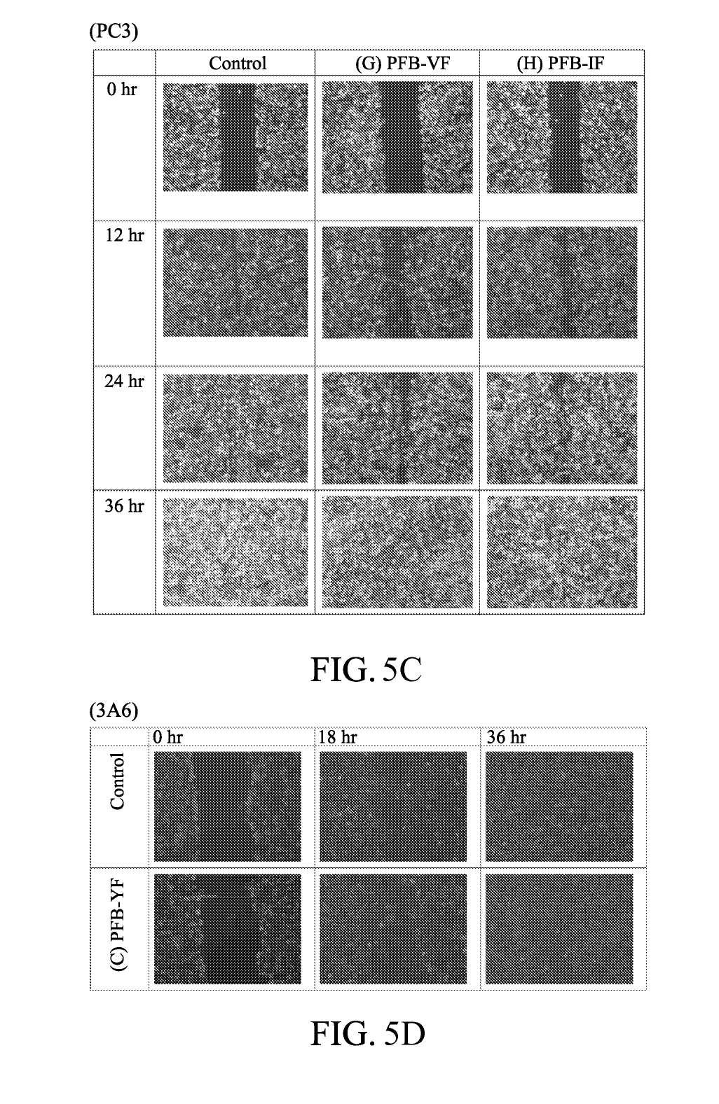

FIG. 5A shows the optical images obtained from the wound healing assays of hydrogels (A), (B), (C), (E), (F) and Control (the tested cell: Hela cell). FIG. 5B shows the optical images obtained from the wound healing assay of hydrogel (C) and Control (the tested cell: CTX TNA2 cell). FIG. 5C shows the optical images obtained from the wound healing assays of hydrogel (G), (H) and Control (the tested cell: PC3 cell). FIG. 5D shows the optical images obtained from the wound healing assays of hydrogel (C) and Control (the tested cell: 3A6 cell). From the wound healing assay, it was found that the effects of hydrogels (A), (B), (C), (E), (F) on the wound healing of Hela cell were similar to that of Control after 24 hours, and that the wounds were healed completely after 48 hours. In addition to Hela cell, PFB-based hydrogels exhibited a better adhesion ability (compared with Hela cell) on other useful cells that have been generally studied, such as CTX TNA2 cell. Further, the result from the wound healing assay (the tested cell: CTX TNA2 cell) of hydrogel (C) shows that the healing effect of hydrogel (C) was better than that of Control.

### Example 7

### Drug Release

The anticancer drug doxorubicin (DOX) was embedded in hydrogel (C). 1.5 ml of water was added on hydrogel (C). Fluorescence spectrometer was be used every 10 minutes.

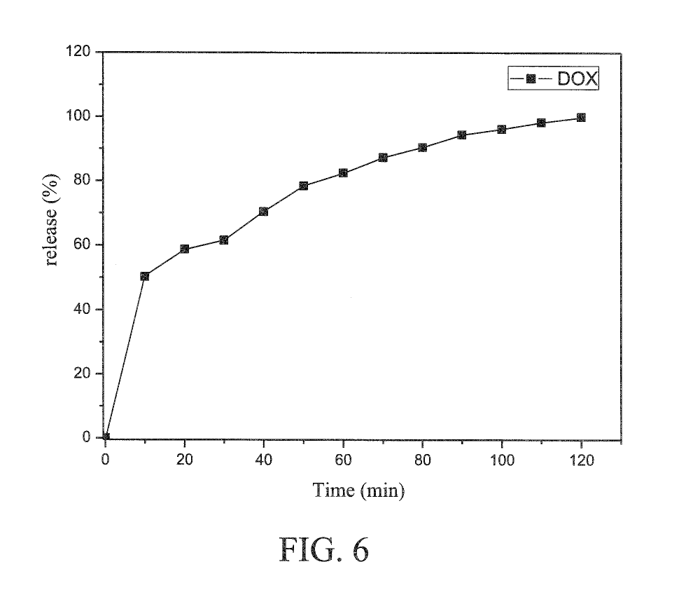

FIG. 6 shows the drug release assay of hydrogel (C) containing the anticancer drug doxorubicin (DOX). It was found that after 20 minutes, the drug was released continuously from the hydrogel. 80% of the drug was release after 1 hour and the drug was released completely within 2 hours. is the result shows that such hydrogel was capable of releasing a drug rapidly.

### Example 8

### 3D Cell Culture

A hydrogel was prepared before cells were seeded. The gelatinization of hydrogel (C) was carried out by adding 0.18 mL of solvent to a vial (2 mL) containing 2.0 mg of PFB-YF compound and adding an alkaline solution until the compound was dissolved. The solution was transferred to a 96-well plate (40 μL/well). An acid solution was added to form the hydrogel in a neutral condition. Subsequently, the hydrogel was placed in an incubator (37° C. and 5% CO₂) overnight for stabilization. Hydrogel (H) were performed based on the same steps. After that, the cells in a concentration of 10000 cells/well were seeded in the 96-well plate which was covered by the hydrogel and contained 0.1 mL of DMEM (Dulbecco's modified eagle medium) with 10% FBS and 1% penicillin. The viability was measured by Live/Dead Viability Assay (molecular probe). On the second day, the cells were washed by PBS twice, were placed in a PBS solution containing 2 μM calcein AM (kit component A) and 4 μM ethidium homodimer-1 (kit component B) and were incubated in an incubator (37° C. and 5% CO₂) for 45 minutes. The cells were washed by PBS several times and were remained in PBS until the image was taken. The data of inverted fluorescence spectrogram were obtained by Zeiss laser scanning microscope. FITC filter: excitation: 440 to 520 nm, emission collection: 510 nm long pass. Rhodamine filter: excitation: 515 to 575 nm, emission collection: 572 nm long pass. The image was combined from FITC filter, Rhodamine filter and bright-field.

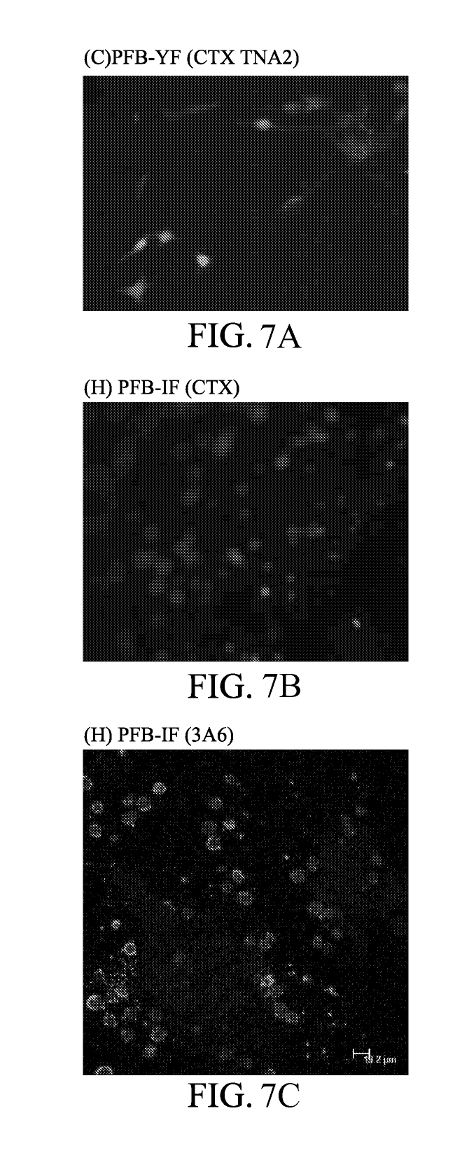

FIG. 7A shows the result obtained from the 3D cell culture of hydrogel (C) (the tested cell: CTX TNA2 cell). FIG. 7B shows the result of 3D cell culture of hydrogel (H) (the tested cell: CTX). FIG. 7C shows the result of 3D cell culture of hydrogel (H) (the tested cell: 3A6). From the result, it was found that the morphology of cells which was cultured on 3D cell culture of the self-assembled hydrogel of the present invention was similar to that of normal CTX TNA2 cell, and no red-dyed cells (i.e., dead cells) were observed. Further, compared with the known techniques, the peptide material of the present invention did not require chemical cross-linker. Also, the peptide material of the present invention was easier to be metabolized, compared with other high molecular materials. The result shows that the peptide material of the present invention is a novel peptide hydrogel material which has the potential to be used in tissue repair.

The above experiments demonstrate that, in addition to non-biotoxicity, the hydrogel of the present invention also has excellent effects on wound healing, drug release and 3D cell culture.

What is claimed is:

1. A peptide molecular material having a structure represented by the following formula 1: 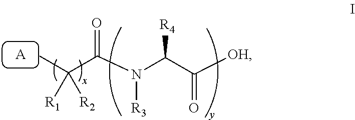
wherein A is phenyl unsubstituted or substituted by one to five halogens, and each of the halogens is selected from the group consisting of fluorine, chlorine, bromine and iodine;
R₁ and R₂ are independently selected from the group consisting of a hydrogen atom and alkyl, and R₁ and R₂ are the same or different;
R₃ and R₄ are independently selected from the group consisting of a hydrogen atom, C₁-C₁₀ alkyl, C₇-C₁₀ aralkyl, C₂-C₁₀ alkylthioalkyl, C₇-C₁₀ hydroxyaralkyl, C₆-C₁₀ heteroaralkyl, C₂-C₁₀ carboxylalkyl, C₂-C₁₀ guanidinoalkyl and C₁-C₁₀ aminoalkyl, and R₃ and R₄ may be the same or different;
x is an integer of 0 to 10, and when x >1, R₁s or R₂s in formula 1 are the same or different; and
y is an integer of 1 to 20, and when y >1, R₃s or R₄s in formula 1 are the same or different.

2. The peptide molecular material according to claim 1, wherein A is phenyl substituted by fluorine; R₁ and R₂ are hydrogen atoms; R₃ and R₄ are independently selected from a hydrogen atom and C₇-C₁₀ aralkyl; x is 1; and y is 1.

3. The peptide molecular material according to claim 1, wherein A is phenyl substituted by fluorine; R₁ and R₂ are independently selected from a hydrogen atom; R₃ and R₄ are independently selected from the group consisting of a hydrogen atom, C₁-C₁₀ alkyl, C₇-C₁₀ aralkyl and C₇-C₁₀ hydroxyaralkyl; x is 1; and y is 2.

4. The peptide molecular material according to claim 1, wherein A is phenyl substituted by fluorine; R₁ and R₂ are independently selected from a hydrogen atom; R₃ and R₄ are independently selected from the group consisting of a hydrogen atom and C₇-C₁₀ aralkyl; x is 1; and y is 3.

5. The peptide molecular material according to claim 1, wherein A is phenyl substituted by fluorine; R₁ and R₂ are hydrogen atoms; R₃ and R₄ are independently selected from the group consisting of a hydrogen atom, C₁-C₁₀ alkyl and C₁-C₁₀ aminoalkyl; x is 1; and y is 5.

6. The peptide molecular material according to claim 1, wherein A is phenyl; R₁ and R₂ are hydrogen atoms; R₃ and R₄ are independently selected from a hydrogen atom, C₁-C₁₀ alkyl and C₁-C₁₀ aminoalkyl; x is 0 to 2; and y is 5.

7. The peptide molecular material according to claim 1, wherein A is phenyl substituted by fluorine; R₁ and R₂ are hydrogen atoms; R₃ and R₄ are independently selected from the group consisting of a hydrogen atom, C₁-C₁₀ alkyl, C₇-C₁₀ aralkyl and C₁-C₁₀ aminoalkyl; x is 1; and y is 6.

8. A self-assembled hydrogel, comprising the peptide molecular material according to claim 1 and water.

9. The self-assembled hydrogel according to claim 8, wherein the pH of the self-assembled hydrogel is in a range of between 5 and 10.

10. The self-assembled hydrogel according to claim 8, having a reticular structure.
